(19) United States
(12) Patent Application Publication
(10) Pub. No.: US 2020/0199141 A1
(43) Pub. Date: Jun. 25, 2020

(54) PSEUDO TRÖGER'S BASE-DERIVED DIANHYDRIDES AND POLYIMIDES DERIVED FROM PSEUDO TRÖGER'S BASE-DERIVED DIANHYDRIDES

(71) Applicant: King Abdullah University of Science and Technology, Thuwal (SA)

(72) Inventors: Xiaohua MA, Thuwal (SA); Mahmoud ABDULHAMID, Thuwal (SA); Ingo PINNAU, Thuwal (SA)

(21) Appl. No.: 16/630,585

(22) PCT Filed: Jun. 12, 2018
(86) PCT No.: PCT/IB2018/054261

§ 371 Date: Jan. 13, 2020

Related U.S. Application Data
(60) Provisional application No. 62/531,465, filed on Jul. 12, 2017.

(51) Int. Cl. C07D 493/04; B01D 53/22; B01D 71/64; B01D 69/02; C07D 493/22; C08G 73/10

(52) U.S. Cl. CPC C07D 493/04; B01D 53/228; B01D 71/64; B01D 2325/20; C07D 493/22; C08G 73/1067; B01D 2325/02; B01D 69/02

(57) ABSTRACT

Embodiments of the present disclosure describe pseudo Tröger's base-derived dianhydrides. Embodiments of the present disclosure also describe polyimides based on pseudo Tröger's base-derived dianhydrides, including intrinsically microporous polyimides. Embodiments of the present disclosure further describe a method of separating chemical species in a fluid composition comprising contacting a polymeric membrane with a fluid composition including at least two chemical species, wherein the polymeric membrane includes one or more of an intrinsically microporous polyimide, and capturing at least one of the chemical species from the fluid composition.

### BACKGROUND

At least one challenge to designing suitable microporous polymers for high-performing polymer-based gas separation membranes is that it is difficult to fabricate polymers that exhibit both high permeability and high selectivity. The empirical Robeson upper bound relationships define an inverse relationship between permeability and selectivity for polymeric membranes. For example, high permeability may be achieved at the cost of selectivity. One solution to overcoming this challenge and designing suitable microporous polymers is to achieve higher gas permeability by increasing the polymer's free volume (e.g., increased chain separation) and to achieve higher selectivity by increasing the polymer's rigidity.

Polymers of intrinsic microporosity (PIM) are one example of polymeric materials that possess high free volume due to contorted and rigid macromolecular chain architectures, which desirably promote inefficient packing and chain rigidity, making them attractive for high-performing polymer-based membranes for gas separation applications. Accordingly, PIMs have attracted significant attention as high-performance materials for a variety of applications, such as gas storage, catalysis, sensors and membranes for gas and liquid separations.

The first PIMs were composed of ladder-type structures connected by rigid contortion sites based on spirobisindane building blocks that generated large amounts of free volume by preventing the polymer main chains from close packing. These amorphous, glassy ladder polymers are generally characterized by: (i) high free volume with internal surface area of up to ˜1000 m²/g and micropores <2 nm, (ii) high thermal stability, (iii) good solution processibility, and (iv) high gas permeability with moderate gas-pair selectivity. More recently, ladder-type Tröger's base (TB) PIMs made from ethanoanthracene—(PIM-EA-TB), spirobisindane—(PIM-SBI-TB), triptycene—(PIM-TRIP-TB), and benzotriptycene—(PIM-BTRIP-TB) diamine building blocks were developed. These PIMs demonstrated substantially enhanced permeability/selectivity performance for a variety of gas pairs, defining the 2015 gas separation performance upper bound curves for O₂/N₂, H₂/N₂, and H₂/CH₄.

Tröger's base-derived diamines were also successfully introduced as PIM-motif building blocks for the synthesis of intrinsically microporous polyimides (PIM-PIs). Some TB-PIM-PIs demonstrated good potential for CO₂/CH₄ separation with performance exceeding the 2008 Robeson upper bound curve. All TB-PIM-PIs reported to date were made from a series of Tröger's base diamines Interestingly, TB- or TB-like dianhydrides have not been reported as alternative building blocks for the synthesis of TB-PIM-PIs.

Accordingly, it would be desirable to provide pseudo TB-derived dianhydrides as building blocks for the synthesis of polyimides.

### SUMMARY

In general, embodiments of the present disclosure describe pseudo TB-derived dianhydrides, polymers based on pseudo TB-derived dianhydrides, and methods of separating fluids using polymer membranes fabricated from polymers based on pseudo TB-derived dianhydrides.

Accordingly, embodiments of the present disclosure describe a pseudo TB-derived dianhydride characterized by one or more of the following chemical structures:

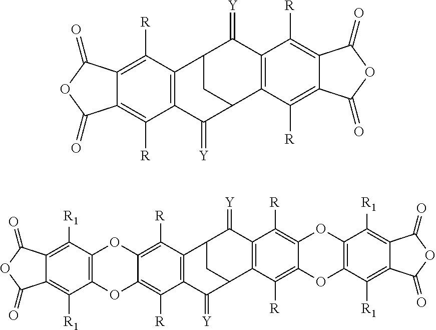

where Y is O, CH₂, or H₂ and each R and R₁ is independently any aromatic group or any aliphatic group.

Embodiments of the present disclosure also describe a polyimide comprising an intrinsically microporous polyimide characterized by one or more of the following chemical structures:

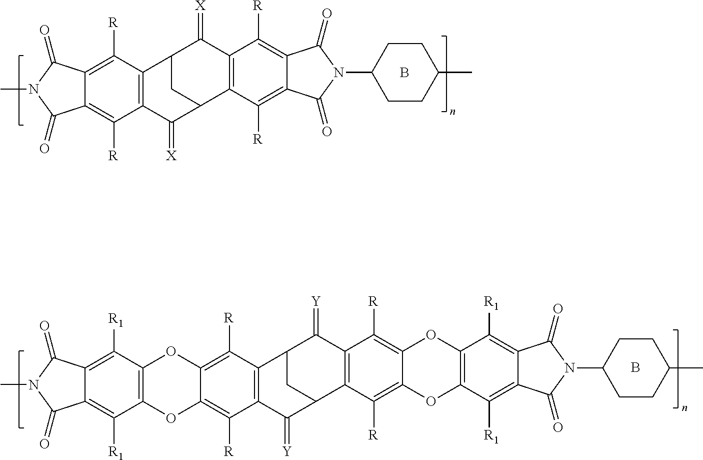

where Y is O, CH₂, or H₂; each R and R₁ is independently any aromatic group or aliphatic group; B is any diamine; and n ranges from 1 to, 10,000.

Embodiments of the present disclosure further describe a polyimide comprising a PIM-PI characterized by one or more of the following chemical structures:

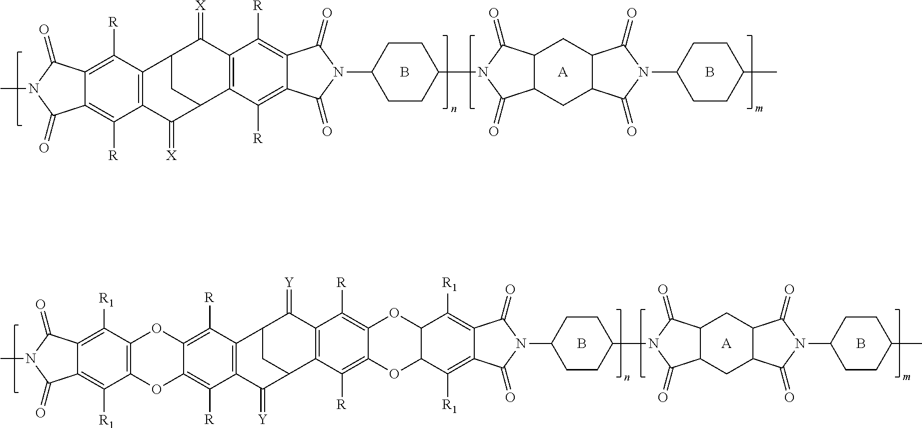

where Y is O, CH₂, or H₂; each R and R₁ is independently any aromatic group or aliphatic group; A is any dianhydride; B is any diamine; and n and m range from 1 to 10,000.

Another embodiment of the present disclosure describes a method of separating chemical species in a fluid composition comprising contacting a polymeric membrane with a fluid composition including at least two chemical species, wherein the polymeric membrane includes a polyimide characterized by one or more of the following chemical structures:

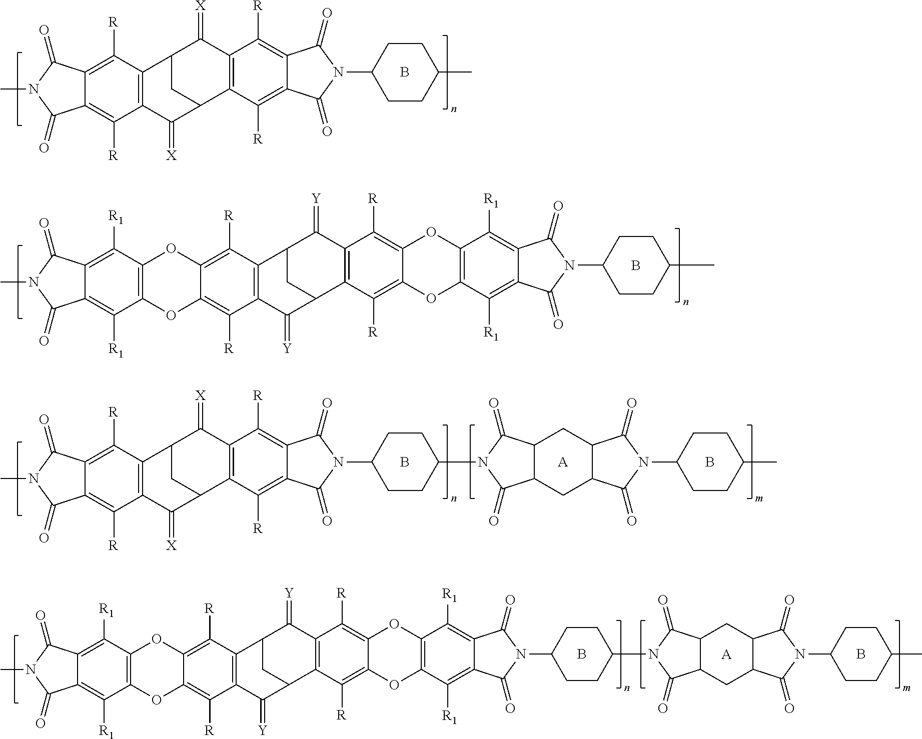

and capturing at least one of the chemical species from the fluid composition.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of synthesizing a polyimide, according to one or more embodiments of the present disclosure.

FIG. 2 is a flowchart of a method of capturing a chemical species, according to one or more embodiments of the present disclosure.

FIG. 3 is ¹H NMR of the CTB1 and CTB2 using deuterated chloroform as solvent, according to one or more embodiments of the present disclosure.

FIG. 4 is TGA of CTB1-DMN and CTB2-DMN (the polymer film samples were heated under N₂ atmosphere at a rate of 3° C./min from room temperature to 800° C.), according to one or more embodiments of the present disclosure.

FIG. 5 is N₂ adsorption/desorption isotherms of CTB1-DMN (blue) and CTB2-DMN (red) at −196° C., according to one or more embodiments of the present disclosure.

FIG. 6 is a graphical view of pore size distribution based on nitrogen adsorption showing incremental volume (cc g⁻¹ Å⁻¹) versus pore width (Å) for CTB1-DMN and CTB2-DMN, according to one or more embodiments of the present disclosure.

FIG. 7 is a graphical view of CO₂ volume adsorbed (cc(STP)/g) versus P/1³. for CTB1-DMN and CTB2-DMN, according to one or more embodiments of the present disclosure.

FIG. 8 is a graphical view of pore size distribution based on CO₂ adsorption showing incremental volume (cc g⁻¹ Å⁻¹) versus pore width (A) for CTB1-DMN and CTB2-DMN, according to one or more embodiments of the present disclosure.

FIG. 9 is UV-vis spectra of the CTB1-DMN and CTB2-DMN polyimide films (10 μm thickness), according to one or more embodiments of the present disclosure.

FIGS. 10a-10b are graphical views illustrating (a) O₂/N₂ and (b) H₂/CH₄ permeability/selectivity performance upper bound plots for PIM-PIs based on 3,3′-dimethylnaphthidine (DMN) and CTB-, TDA-, EADA-, SBFDA- and SBIDA-dianhydrides for fresh and aged samples, according to one or more embodiments of the present disclosure.

### DETAILED DESCRIPTION

The invention of the present disclosure relates to novel pseudo Tröger's Base (TB)-derived dianhydrides and novel microporous polyimides derived from the pseudo Tröger's Base-derived dianhydrides. The pseudo TB-derived dianhydrides of the present disclosure may be carbocyclic pseudo TB-derived dianhydrides. The pseudo TB-derived dianhyrides may be used as building blocks for the synthesis of various microporous polyimides. The high molecular weight of the microporous polyimides derived from these pseudo TB-derived dianhydrides is similar to conventional polyimides. In addition, unlike many conventional polyimides, the microporous polyimides of the present disclosure are soluble in common organic solvents. Accordingly, the microporous polyimides of the present disclosure may be used to fabricate polymer membranes (e.g., thin films) with excellent fluid transport properties (e.g., gas transport properties). In this way, the invention of the present disclosure describes dianhydrides and microporous polymers that may be used to fabricate membranes suitable for a wide variety of membrane-based fluid separation applications, including, but not limited to, fluid separations such as air separation, hydrogen/methane separation, hydrogen/nitrogen separation, hydrogen/carbon monoxide separation, CO₂ and H₂S removal from natural gas, olefin/paraffin separation, and dehydration of air and natural gas.

As one example, the invention of the present disclosure relates to two novel carbocyclic pseudo Tröger's base-derived dianhydrides, 5,6,11,12-tetrahydro-5,11-methanodibenzo[a,e][8]annulene-2,3,8,9-tetracarboxylic anhydride (CTB1) and its dione-substituted analogue, 6,12-dioxo-5,6,11,12-tetrahydro-5,11-methanodibenzo[a,e][8]annulene-2,3,8,9-tetracarboxylic dianhydride (CTB2), as well as the synthesis, general physical properties, and gas performance of PIM-PIs made from CTB1 and CTB2. CTB1 and CTB2 were made and used for the synthesis of soluble polyimides of intrinsic microporosity with 3,3′-dimethylnaphthidine (DMN). The polyimides CTB1-DMN and CTB2-DMN exhibited excellent thermal stability of ˜500° C. and high BET surface areas of 580 and 469 m² g⁻¹, respectively. A freshly made dione-substituted CTB2-DMN membrane demonstrated promising gas separation performance with O₂ permeability of 206 Barrer and O₂/N₂ selectivity of 5.2. A higher O₂ permeability of 320 Barrer and lower O₂/N₂ selectivity of 4.2 was observed for a fresh CTB1-DMN film due to its higher surface area and less tightly packed structure as indicated by weaker charge-transfer complex interactions. Physical aging over 60 days resulted in reduction in gas permeability and moderately enhanced selectivity. CTB2-DMN exhibited notable performance with gas permeation data located between the 2008 and 2015 permeability/selectivity upper bounds for O₂/N₂ and H₂/CH₄.

### Definitions

The terms recited below have been defined as described below. All other terms and phrases in this disclosure shall be construed according to their ordinary meaning as understood by one of skill in the art.

As used herein, “aliphatic” refers to organic compounds and/or radicals characterized by substituted or un-substituted straight, branched, and/or cyclic chain arrangements of constituent carbon atoms. Carbon atoms may be joined by single bonds, double bonds, or triple bonds. The term “aliphatic” includes cycloaliphatic compounds/groups and/or alicyclic compounds/groups.

As used herein, “aromatic” refers to aromaticity, a chemical property in which a conjugated ring of unsaturated bonds, lone pairs, or empty orbitals exhibit a stabilization stronger than would be expected by the stabilization of conjugation alone.

As used herein, “capturing” refers to the act of removing one or more chemical species from a bulk fluid composition (e.g., gas/vapor, liquid, and/or solid). For example, “capturing” may include, but is not limited to, interacting, bonding, diffusing, adsorbing, absorbing, reacting, and sieving, whether chemically, electronically, electrostatically, physically, or kinetically driven.

As used herein, “contacting” refers to the act of touching, making contact, or of bringing to immediate or close proximity, including at the molecular level, for example, to bring about a chemical reaction, or a physical change, e.g., in a solution, in a reaction mixture. Accordingly, adding, stirring, treating, tumbling, vibrating, shaking, mixing, and applying are forms of contacting to bring two or more components together.

As used herein, “contacting” may, in the alternative, refer to, among other things, feeding, flowing, passing, injecting, introducing, and/or providing the fluid composition (e.g., a feed gas).

As used herein, “anhydride” refers to a moiety of the formula R₁-C(═O)—O—C(═O)—R₂, where R₁ and R2 are independently alkyl, haloalkyl, aryl, cycloalkyl, aromatic alkyl, (cycloalkyl)alkyl and the like.

As used herein, “aryl group” refers to a monovalent mono-, bi- or tricyclic aromatic hydrocarbon moiety of 6 to 15 ring atoms, which is optionally substituted with one or more, typically one, two, or three substituents within the ring structure. When two or more substituents are present in an aryl group, each substituent is independently selected. Exemplary aryl includes, but is not limited to, phenyl, 1-naphthyl, and 2-naphthyl, and the like, each of which can optionally be substituted.

As used herein, “alkyl group” refers to a functional group including any alkane with a hydrogen removed therefrom. For example, “alkyl” may refer to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl groups include, but are not limited to, methyl, ethyl, 1-propyl, 2-propyl, tert-butyl, pentyl, and the like.

As used herein, “carbocyclic” refers to a cyclic arrangement of carbon atoms forming a ring. The term “carbocyclic” may be distinguished from heterocyclic rings in which the ring backbone contains at least one atom which is different from carbon.

As used herein, “halogen” refers to any elements classified as halogens according to the Periodic Table. Halogens may include one or more of fluorine, chlorine, bromine, and iodine.

As used herein, “heteroaryl group” refers to a monovalent mono- or bicyclic aromatic moiety of 5 to 12 ring atoms containing one, two, or three ring heteroatoms selected from N, O, or S, the remaining ring atoms being C. The heteroaryl ring can be optionally substituted with one or more substituents, typically one or two substituents. Exemplary heteroaryl includes, but is not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

### Pseudo TB-Derived Dianhydrides

Embodiments of the present disclosure describe a pseudo TB-derived dianhydride. In many embodiments, the pseudo TB-derived dianhyride may be a carbocyclic pseudo TB-derived dianhydride. For example, the carbocyclic pseudo TB-derived dianhydride may be characterized by one or more of the following chemical structures:

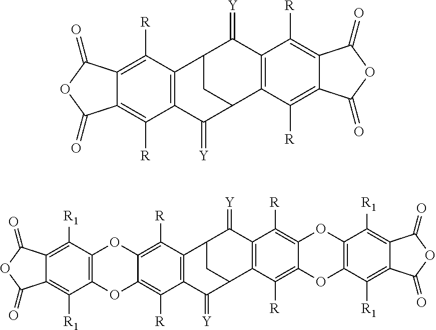

where Y is O, CH₂, or H₂ and each R and R₁ is independently hydrogen or any hydrocarbon. In many embodiments, each R and R₁ may independently be any aromatic group or any aliphatic group. For example, each R and R₁ may independently include one or more of methyl, ethyl, propyl, isopropyl, n-butyl, and iso-butyl.

As one example, the carbocyclic pseudo TB-derived dianhydride may include 5,6,11,12-tetrahydro-5,11-methanodibenzo[a,e][8]annulene-2,3,8,9-tetracarboxylic dianhydride (CTBA1). CTBA1 may be characterized by the following chemical structure:

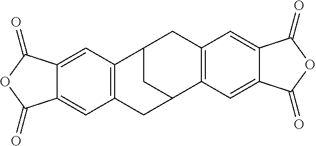 [SMILES: O=C1OC(=O)c2cc3c(cc21)CC1CC3Cc2cc3c(cc21)C(=O)OC3=O]

As another example, the carbocyclic pseudo TB-derived dianhydride may include 6,12-dioxo-5,6,11,12-tetrahydro-5,11-methanodibenzo[a,e][8]annulene-2,3,8,9-tetracarboxylic dianhydride (CTB2), which is the dione-substituted analog of CTB1. CTB2 may be characterized by the following chemical structure:

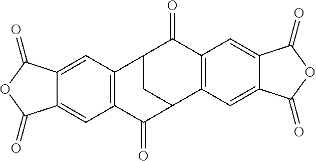 [SMILES: O=C1OC(=O)c2cc3c(cc21)C(=O)C1CC3C(=O)c2cc3c(cc21)C(=O)OC3=O]

The synthetic route for synthesizing pseudo TB-derived dianhydride CTB1 followed the method outlined in PCT/IB2016/056778, which is hereby incorporated by reference in its entirety. Here, the hydrolysis of the CTB1-tetracyano intermediate was done by reaction with KOH/H₂O. In case of CTB2, a modification in the hydrolysis of the tetracyano intermediate involved a reaction with H₂SO₄/H₂O. Schemes 1a and 1b are examples of synthetic routes for synthesizing pseudo TB-derived dianhydrides from biscatechols.

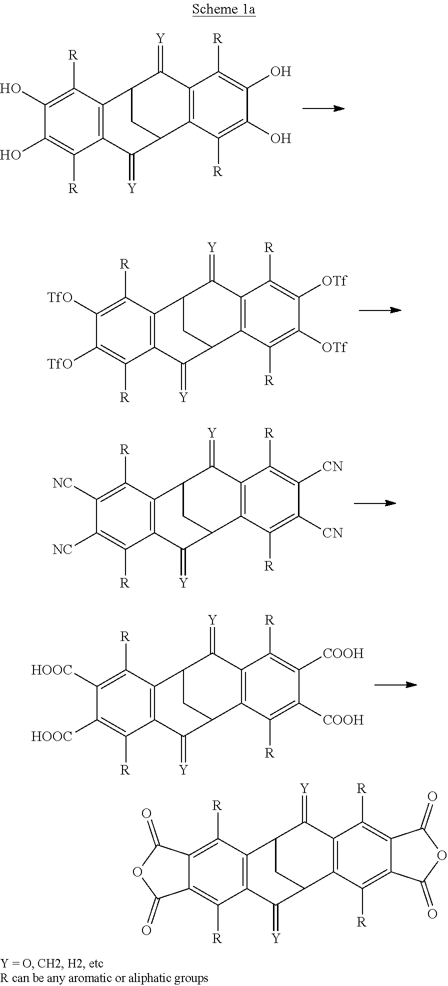

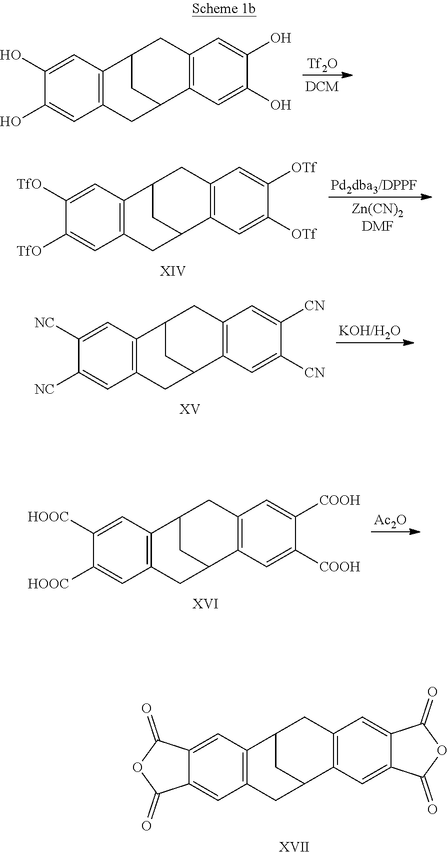

The biscatechols from which the pseudo TB-derived dianhydrides are synthesized may include one or more of the following chemical structures:

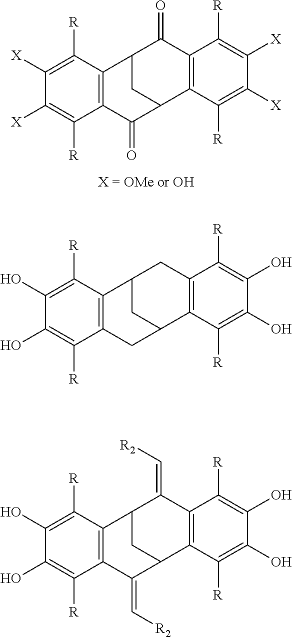

where each R and R₂ is independently any aliphatic group or aromatic group.

### Polyimides

The pseudo TB-derived dianhydrides (e.g., the carbocyclic pseudo TB-derived dianhydrides) may be used as a building block for the synthesis of various polyimides. In particular, the pseudo TB-derived dianhydride may be used as a building block for the synthesis of microporous polyimides and polymers of intrinsic microporosity polyimides (PIM-PI). The polyimides based on the carbocyclic pseudo TB-derived dianhydrides exhibit properties that are superior to conventional polyimides. Unlike many conventional polyimides, the polyimides of the present disclosure are soluble (e.g., highly soluble) in organic solvents and have high molecular weights, with narrow polydispersity indexes and excellent thermal stability. For example, the polyimides may be soluble in N-methyl-2-pyrrolidone (NMP), dimethylformamide (DMF), dimethylacetamide (DMAc), m-cresol, chloroform, and other organic solvents known in the art. The molecular weight (e.g., number average molecular weight) of the polyimides may be up to about 60,000 g mol⁻¹. In other embodiments, the molecular weight may range from about 20,000 g mol⁻¹ to about 60,000 g mol⁻¹. Because of their high molecular weights and excellent solubility, the CTB-based polyimides can be cast into mechanically strong films and membranes. The polyimides may exhibit onset decomposition temperatures between about 480° C. and about 520° C. In addition, the membranes based on these polyimides and/or the polyimides of the present disclosure have high microporosity and high BET surface areas. These and numerous other advantages of polyimides based on carbocyclic pseudo TB-derived dianhydrides are discussed further below and elsewhere herein.

Accordingly, embodiments of the present disclosure describe microporous polyimides. In many embodiments, the microporous polyimides may be characterized by one or more of the following chemical structures:

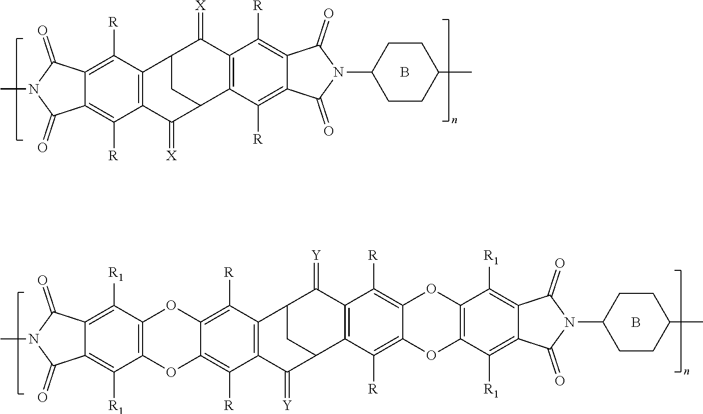

where each X and Y is independently O, CH₂, or H₂; each R and R₁ is independently hydrogen or any hydrocarbon; B has a chemical structure as defined below; and n ranges from 1 to 10,000. In many embodiments, each R and R₁ may independently be any aromatic group or any aliphatic group. For example, each R and R₁ may independently include one or more of methyl, ethyl, propyl, isopropyl, n-butyl, and iso-butyl.

The general characteristic structure of B is derived from aromatic diamines. In many embodiments, the chemical structure for B may be characterized by one or more of the following chemical structures:

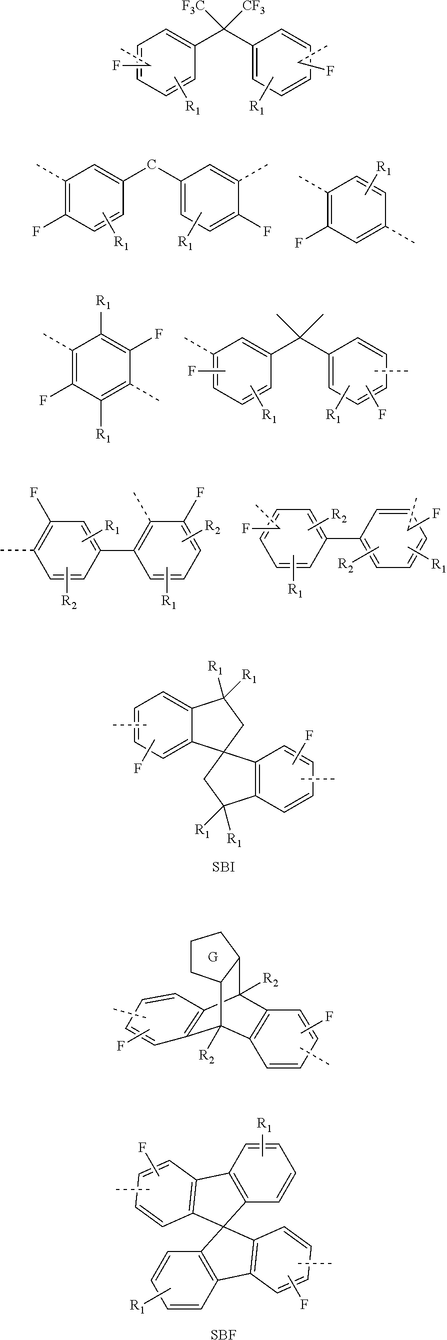

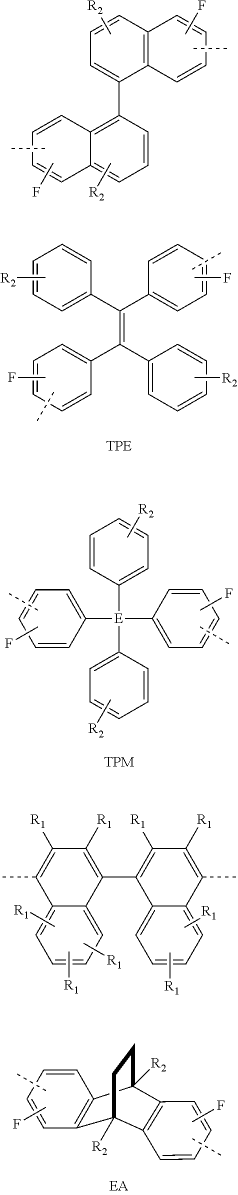

where C is O, S, SO₂, or CH₂; E is C or Si; F is O or OH; G is any substituted phenyl group, thiophenyl group, or furanyl group; and each R₁ and R₂ is independently hydrogen or any hydrocarbon. In many embodiments, each R and R₁ may independently be any aromatic group or any aliphatic group. For example, each R and R₁ may independently include one or more of methyl, ethyl, propyl, isopropyl, n-butyl, and iso-butyl. In other embodiments, each R₁ and R₂ is independently one or more of the following chemical structures:

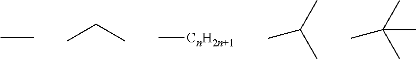

where n is at least 1. In some embodiments, G is characterized by the following chemical structure:

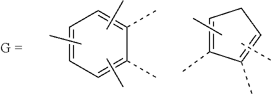

As one example, the microporous polyimide may include 5,6,11,12-tetrahydro-5,11-methanodibenzo[a,e][8]annulene-2,3,8,9-tetracarboxylic dianhydride-dimethylnaphthidine (CTB1-DMN). CTB1-DMN may be characterized by the following chemical structure:

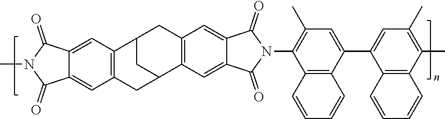

As another example, the microporous polyimide may include 6,12-dioxo-5,6,11,12-tetrahydro-5,11-methanodibenzo[a,e][8]annulene-2,3,8,9-tetracarboxylic dianhydride-dimethylnaphthidine (CTB2-DMN). CTB2-DMN may be characterized by the following chemical structure:

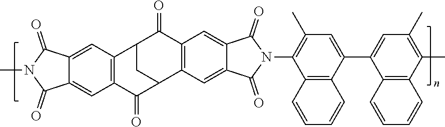

Embodiments of the present disclosure further describe polymers of intrinsic microporosity polyimides (PIM-PI) characterized by one or more of the following chemical structures:

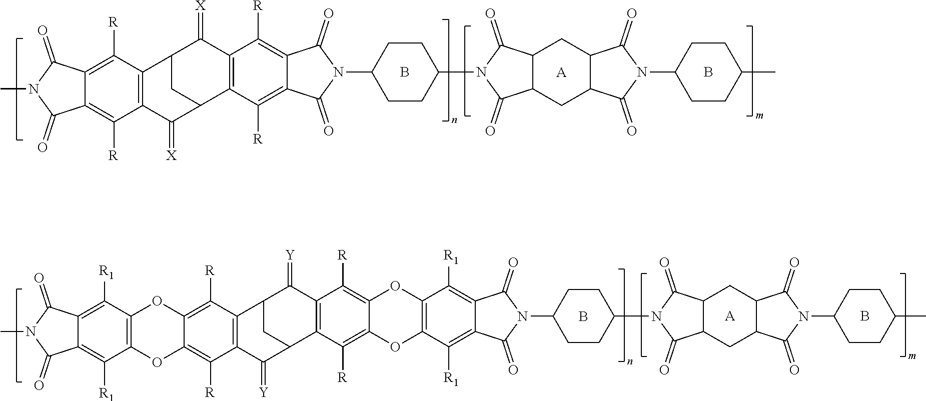

where each X and Y is independently O, CH₂, or H₂; each R and R₁ is independently hydrogen or any hydrocarbon; A has a chemical structure as defined below; B has a chemical structure as defined above, which is hereby incorporated by reference in its entirety; and n and m range from 1 to 10,000. In many embodiments, each R and R₁ may independently be any aromatic group or any aliphatic group. For example, each R and R₁ may independently include one or more of methyl, ethyl, propyl, isopropyl, n-butyl, and iso-butyl.

The general characteristic structure of A is derived from aromatic dianhydrides. In many embodiments, the chemical structure for A may be characterized by one or more of the following chemical structures:

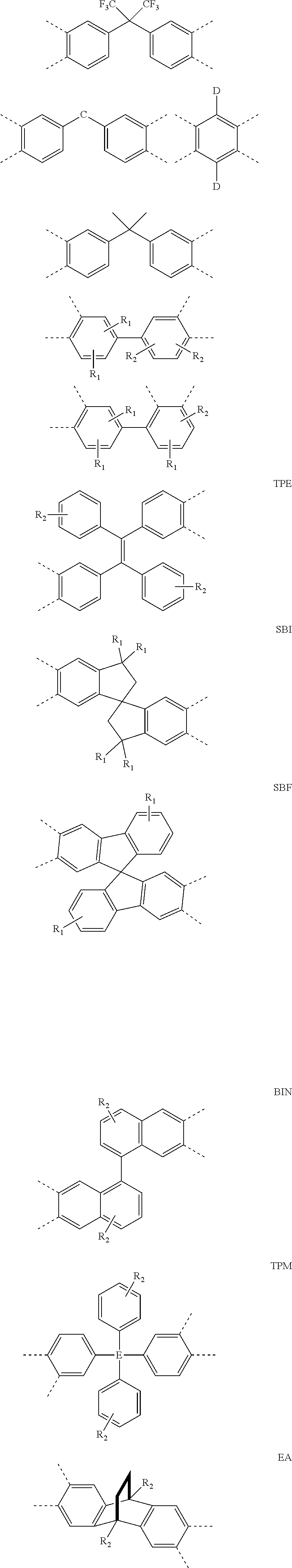

where C is O, S, SO₂, or CH₂; D is H, CH₃, C₂H₅, or CF₃; E is C or Si; F is O or OH; each R₁ and R₂ is independently hydrogen or any hydrocarbon. In many embodiments, each R and R₁ may independently be any aromatic group or any aliphatic group. For example, each R and R₁ may independently include one or more of methyl, ethyl, propyl, isopropyl, n-butyl, and iso-butyl. In other embodiments, each R₁ and R₂ is independently one or more of the following chemical structures:

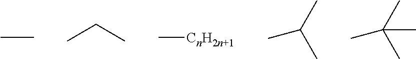

where n is at least 1.

### Synthesis of Polyimides

A general synthetic procedure for synthesizing polyimides is provided as an example in Scheme 2:

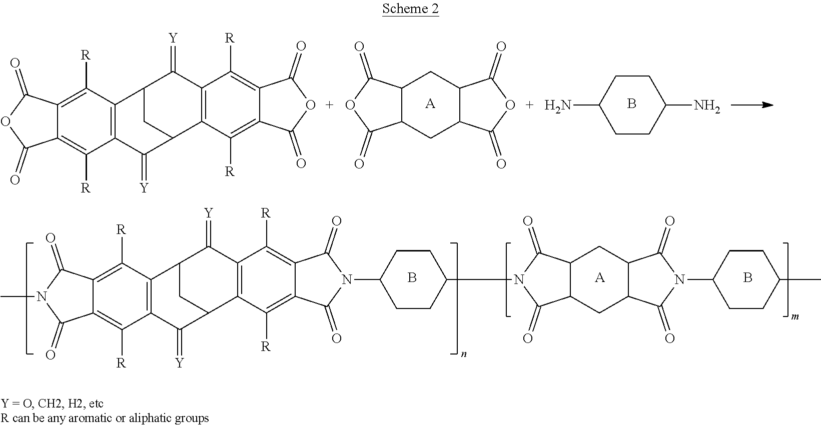

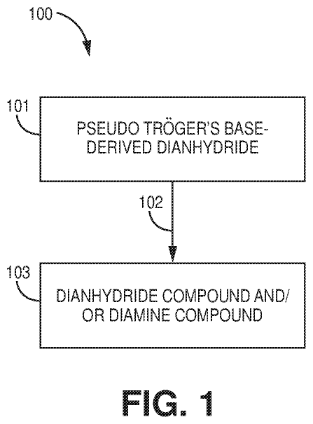

FIG. 1 is a flowchart of a method of synthesizing polyimides, according to one or more embodiments of the present disclosure. As shown in FIG. 1, a pseudo TB-derived dianhydride (101) is contacted with a diamine compound (103) to form a polyimide homopolymer. In another embodiment, a pseudo TB-derived dianhydride (101) is contacted with an aromatic dianhydride (102) and with a diamine compound (103) to form a co-polyimide. The pseudo TB-derived dianhydride may include any of the pseudo TB-derived dianhydrides described above and elsewhere herein, which is hereby incorporated by reference in its entirety. The aromatic dianhydride compound (102) is optional, as a microporous polyimide may be formed in the absence of the aromatic dianhydride compound; and a polymer of intrinsic microporosity may be formed where both the dianhydride compound (101 and 102) and diamine compound (103) are contacted with the pseudo TB-derived dianhydride.

The dianhydride compound 102 may include any dianhydride. In many embodiments, the dianhydride compound may include a tetracarboxylic dianhydride. For example, the dianhydride compound may be characterized by the following chemical structure:

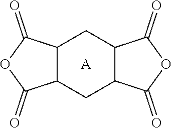

where A is as defined above and elsewhere herein, which is hereby incorporated by reference in its entirety.

The diamine compound 103 may include any diamine In many embodiments, the diamine compound may be characterized by the following chemical structure:

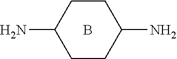

where B is as defined above and elsewhere herein, which is hereby incorporated by reference in its entirety.

Contacting 102 may include adding the pseudo TB-derived dianhydride and one or more of the dianhydride compound and the diamine compound to a solution. In many embodiments, the solution includes one or more of m-cresol and isoquinoline. The temperature of the contacting may range from about room temperature to about 200° C.

The polyimide may include a microporous polyimide. In some embodiments, the pseudo TB-derived dianhydride is contacted with the diamine compound to form a microporous polyimide. In these embodiments, the polyimide may be characterized by one or more of the following chemical structures:

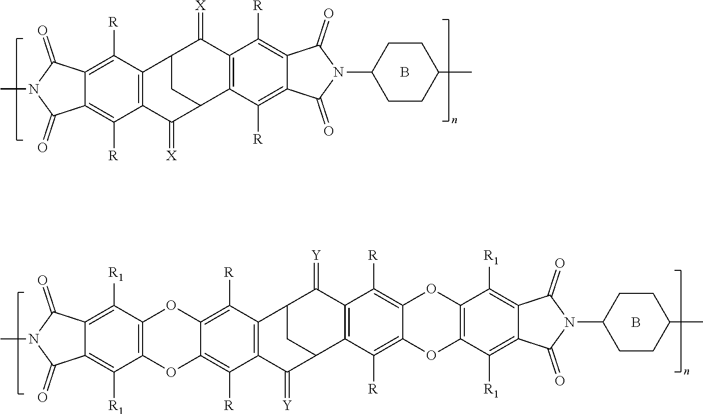

where each X and Y is independently O, CH₂, or H₂; each R and R₁ is independently hydrogen or any hydrocarbon; B is any diamine as defined above and elsewhere herein; and n ranges from 1 to 10,000. In many embodiments, each R and R₁ may independently be any aromatic group or any aliphatic group. For example, each R and R₁ may independently include one or more of methyl, ethyl, propyl, isopropyl, n-butyl, and iso-butyl.

In other embodiments, the pseudo TB-derived dianhydride 101 is contacted with the dianhydride compound 102 and the diamine compound 103 to form a microporous co-polyimide. In these embodiments, the polyimide may be characterized by one or more of the following chemical structures:

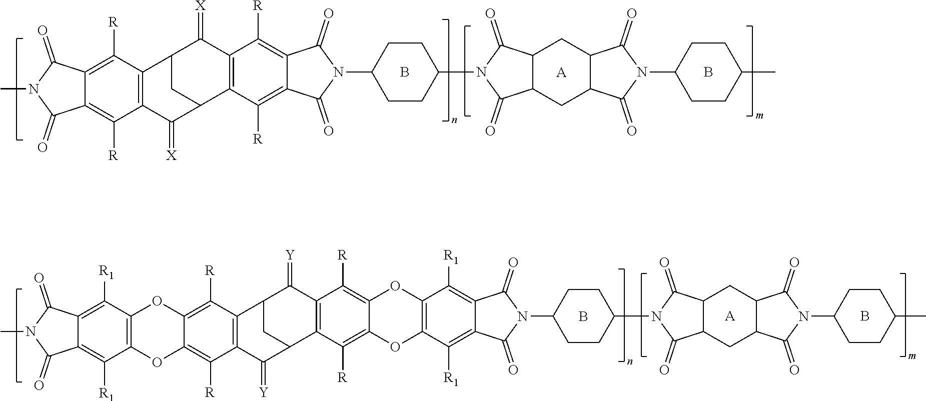

where each of X and Y is independently one or more of O, CH₂, and H; each of R and R₁ is independently any aromatic group or aliphatic group; A is any dianhydride as defined above and elsewhere herein; B is any diamine as defined above and elsewhere herein; and each m and n ranges from 1 to 10,000.

In one embodiment, a pseudo TB-derived dianhydride is contacted with 3,3′-dimethylnaphthidine (DMN) to form a CTB1-DMN polyimide and/or a CTB2-DMN polyimide as described above and elsewhere herein, which is hereby incorporated by reference in its entirety.

### Polymer Membranes for Fluid Separations

The microporous polyimides based on carbocyclic pseudo TB-derived dianhydrides may be used to fabricate polymer membranes (e.g., polymer films) that exhibit excellent fluid transport properties (e.g., gas transport properties). These polymer membranes may include inefficient chain packing and high chain rigidity, with size-selective ultramicropores (about <7 Å). In addition, these polymer membranes may exhibit strong charge-transform complex formation (CTC) and high BET surface areas. The polymer membranes accordingly exhibit high gas permeabilities and moderate to high gas-pair selectivities that exceed and/or approach the upper bounds for numerous gas pairs. The polymer membranes may further exhibit low degradation in response to physical aging.

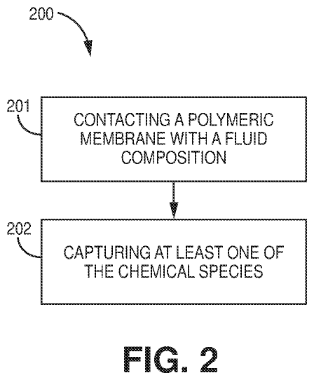

Accordingly, FIG. 2 is a flowchart of a method of separating chemical species in a fluid composition, according to one or more embodiments of the present disclosure. At step 201, a polyimide-based membrane is contacted with a fluid composition including at least two chemical species. At step 202, the polyimide-based membrane captures at least one of the chemical species from the fluid composition.

The polyimide-based membrane may include any of the polymers of the present disclosure. In many embodiments, the polyimide-based membrane may include a microporous polymer. For example, the microporous polymer may include a polyimide (e.g., an intrinsically microporous polyimide, a polymer of intrinsic microporosity polyimide, etc.). In many embodiments, the microporous polymer may be derived from a carbocyclic pseudo TB-derived dianhydride. For example, the carbocyclic pseudo TB-derived dianhydride may be characterized by one or more of the following chemical structures:

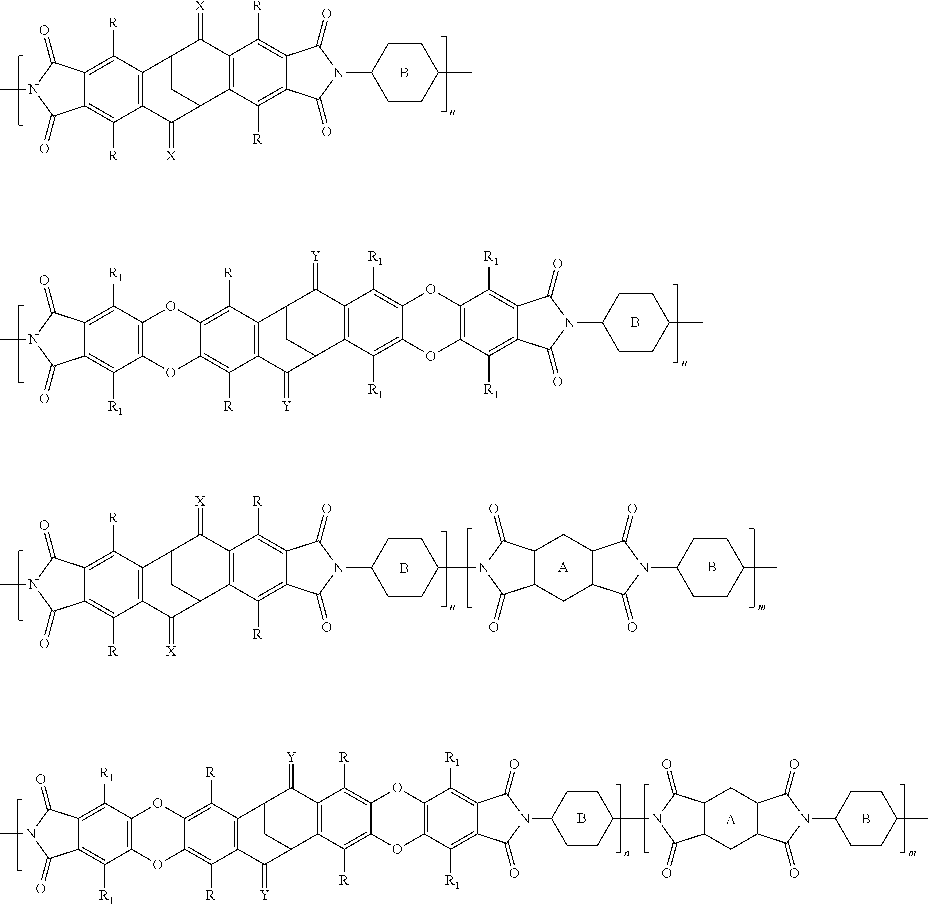

The fluid composition may include chemical species in a gas/vapor phase, liquid phase, solid phase, or any combination thereof. The chemical species of the fluid composition may include one or more of O₂, N₂, H₂, He, CH₄, CO₂, C₂₊ hydrocarbons, olefins, paraffins, n-butane, iso-butane, butenes, and xylene isomers. In many embodiments, the fluid composition includes at least two chemical species. For example, the fluid composition may include at least one or more of the following pairs of chemical species: O₂ and N₂, H₂ and N₂, H₂ and CH₄, CO₂ and CH₄, H₂ and C₂₊ hydrocarbons, He and C₁₊ hydrocarbons, CO₂ and C₂₊ hydrocarbons, CO₂ and N₂, olefins and paraffins, n-butane and iso-butane, n-butane and butenes, xylene isomers, and combinations thereof. In other embodiments, any combination of chemical species may be included and/or present in the fluid composition.

Contacting may refer to, among other things, feeding, flowing, passing, injecting, introducing, and/or providing the fluid composition (e.g., a feed gas). The contacting may occur at various pressures, temperatures, and concentrations of chemical species in the fluid composition, depending on desired feed conditions and/or reaction conditions. The pressure, temperature, and concentration at which the contacting occurred may be varied and/or adjusted according to a specific application.

The captured chemical species may include one or more of O₂, N₂, H₂, CH₄, CO₂, and He. In many embodiments, the permeabilities of the polyimide-based membrane may follow the order P_{CH4}<P_{N2}<P_{O2}<P_{H2}<P_{CO2}. In other embodiments, the permeabilities of the polyimide-based membrane may follow the order of P_{CH4}˜P₂<P_{O2}<P_{H2}<P_{CO2}. In embodiments in which the fluid composition includes H₂ and N₂, the captured chemical species may include H₂. In embodiments in which the fluid composition includes H₂ and CH₄, the captured chemical species may include H₂. In embodiments in which the fluid composition includes O₂ and N₂, the captured chemical species may include O₂. In embodiments in which the fluid composition includes CO₂ and CH₄, the captured chemical species may include CO₂.

Capturing may refer to the act of removing one or more chemical species from a bulk fluid composition (e.g., gas/vapor, liquid, and/or solid). The capturing of the one or more chemical species may depend on a number of factors, including, but not limited to, selectivity, diffusivity, permeability, solubility, conditions (e.g., temperature, pressure, and concentration), membrane properties (e.g., pore size), and the methods used to fabricate the membranes.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### EXAMPLE 1

### CTB1-DMN and CTB2-DMN

This Example describes for the first time two carbocyclic pseudo TB-derived dianhydrides, 5,6,11,12-tetrahydro-5,11-methanodibenzo [a,e][8]annulene-2,3,8,9-tetracarboxylic anhydride (CTB1) and its dione-substituted analogue 6,12-dioxo-5,6,11,12-tetrahydro-5,11-methanodibenzo[a,e][8]annulene-2,3,8,9-tetracarboxylic dianhydride (CTB2). This Example further describes the synthesis, general physical properties, and gas separation performance of PIM-PIs made from CTB1 and CTB2 with dimethylnaphthidine (DMN).

Examples of the synthetic procedure for the carbocyclic pseudo TB-derived dianhydrides (CTB1 and CTB2) and corresponding PIM-PIs (CTB1-DMN and CTB2-DMN) is shown below in Scheme 3:

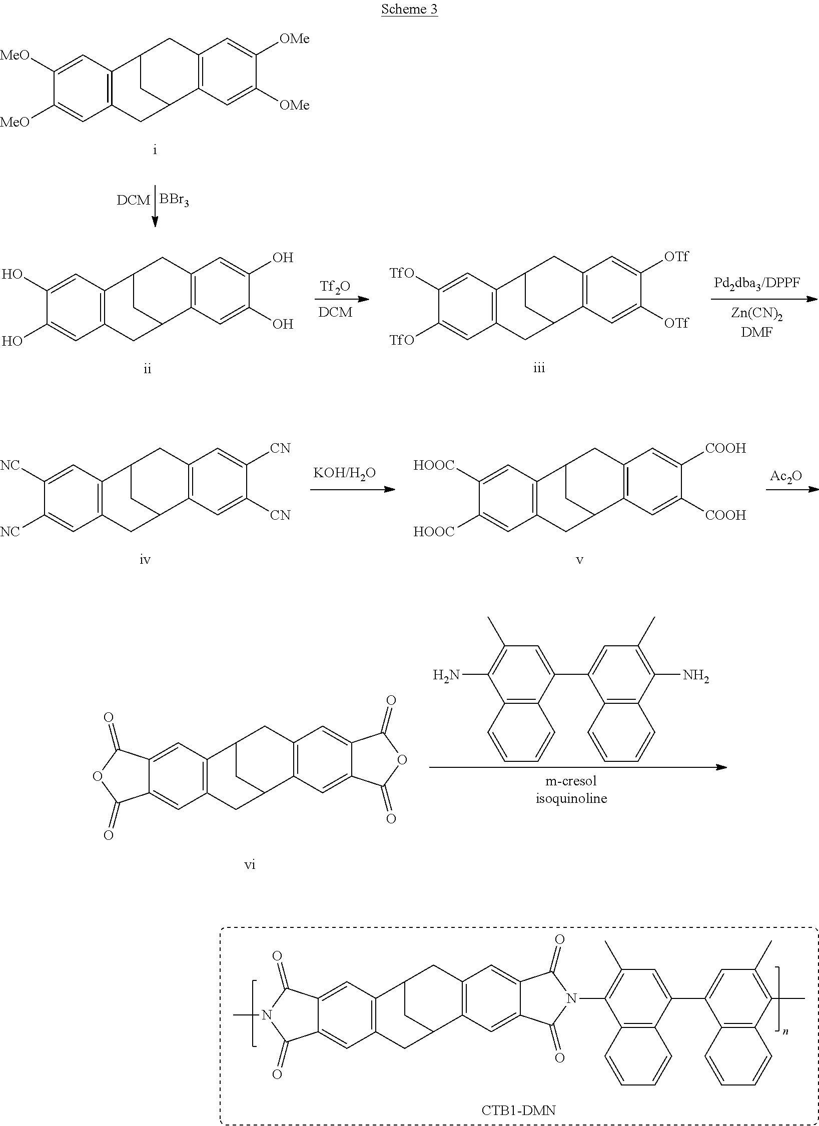

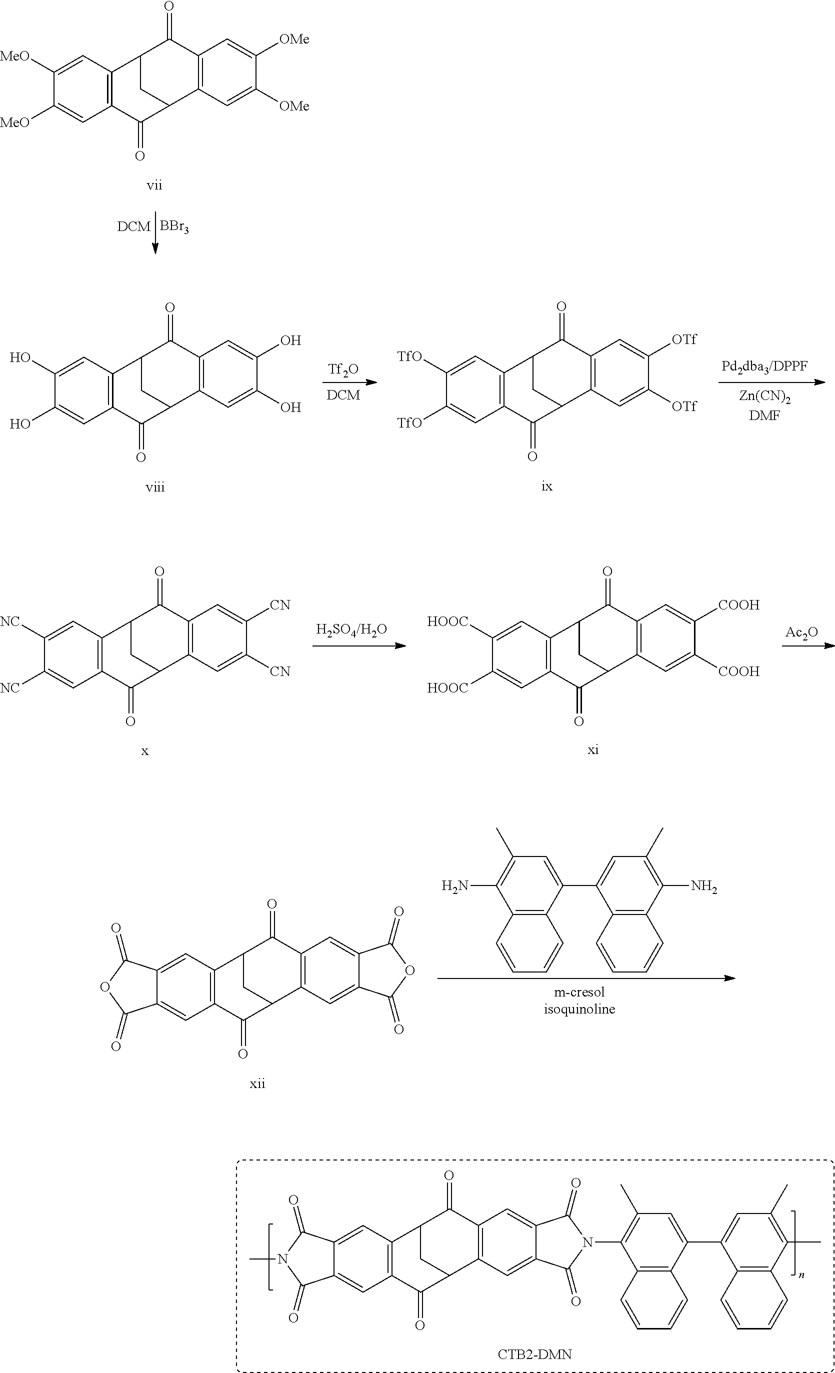

### Experimental

Materials. 6,12-Dioxo-5,6,11,12-tetrahydro-5,11-methanodibenzo[a,e][8]annulene-2,3,8,9-tetramethyoxylether, 5,6,11,12-tetrahydro-5,11-methanodibenzo[a,e][8]annulene-2,3,8,9-tetramethoxyl ether were synthesized. Trifluoromethane sulfonic anhydride (Tf₂O), dichloromethane, triethylamine, boron tribromide, HCl (12N), tris(dibenzylideneacetone)dipalladium (0) (Pd₂dba₃), 1,1-ferrocenediyl-bis(diphenylphosphine) (DPPF), zinc cyanate (Zn(CN)₂), methanol, N,N-dimethylformamide, concentrated sulfuric acid, acetic anhydride, m-cresol, isoquinoline and silica gel were obtained from Sigma-Aldrich and used as received. 3,3-dimethylnaphthidine (>97% purity) was purchased from TCI and used as received.

Characterization. ¹H NMR and ¹³C NMR spectra of the newly synthesized monomers and polymers were recorded with a Bruker AVANCE-III spectrometer at a frequency of 400 or 500 MHz in either deuterated chloroform or deuterated dimethylsulfone with tetramethylsilane as an internal standard and recorded in ppm. Molecular weight (Mn) and molecular weight distribution (PDI) of CTB1-DMN and CTB2-DMN were obtained by gel permeation chromatography (GPC) (Agilent 1200) using DMF and chloroform as solvent and polystyrene as external standard, respectively. FT-IR of the polyimides were acquired using a Varian 670-IR FT-IR spectrometer. Thermal gravimetric analysis (TGA) was carried out using a TGA Q5000 (TA Instruments); the polymers were heated from room temperature to 800° C. under N₂ atmosphere at a heating rate of 3° C./min. Melting points of the intermediates were obtained by differential scanning calorimetry (DSC, TA Instruments Q2000). UV-vis spectra of the polymer films were recorded using a Lambda 1050 spectrophotometer. The Brunauer-Emmett-Teller (BET) surface area of the polymers was determined by N₂ adsorption at −196° C. (Micrometrics ASAP 2020); each sample was degassed at 150° C. for 12 h before testing. A Mettler-Toledo balance equipped with a density measurement kit was used to determine the polymer density based on Archimedes' principle using iso-octane as the reference liquid.

Synthesis of 5,6,11,12-tetrahydro-5,11-methanodibenzo[a,e][8]annulene-2,3,8,9-tetrayl tetraoh (ii). 5,6,11,12-Tetrahydro-5,11-methanodibenzo [a,e][8]annulene-2,3,8,9-tetramethoxyl (i) (2.00 g, 5.88 mmol) was dissolved in 150 mL dichloromethane and cooled in an ice bath. To it, BBr₃ (1.67 mL, 17.6 mmol) was added to the solution dropwise. After the solution was stirred at room temperature for 24 h and then poured into 200 g crushed ice, an off-white powder was obtained after stirring under N₂ for another 24 h. The resulting intermediate ii was obtained as an off-white solid with a yield of 96%. ¹H NMR (500 MHz, DMSO-d₆): δ 8.47 (s, 4H), 6.47 (s, 2H), 6.22 (s, 2H), 2.94 (t, 4H, J=7.00 Hz, 9.17 Hz), 2.40 (d, 2H, J=11.8 Hz), 1.83 (s, 2H).

Synthesis of 5,6,11,12-tetrahydro-5,11-methanodibenzo[a,e][8]annulene-2,3,8,9-tetrayl tetrakis(trifluoromethanesulfonate) (iii). 5,6,11,12-Tetrahydro-5,11-methanodibenzo [a,e][8]annulene-2,3,8,9-tetraoh (2.00 g, 7.04 mmol, ii) and triethylamine (13.76 g, 128.0 mmol) were added to dichloromethane (150 mL) and cooled in an ice bath. To it, triflic anhydride (32.0 g, 128.0 mmol) was added dropwise. The reaction system was further stirred for 12 h and poured into ice water (300 mL). The water phase was then extracted twice with dichloromethane (2×30 mL). The organic phase was combined and dried with magnesium sulfate. The solution was removed by rota-evaporation and loaded to a column packed with silica gel. An off-white product (3.42 g, yield: 60%) was obtained after column chromatography. TLC: dichloromethane, R_{f}=0.5; ¹H NMR (500 MHz, CDCl₃): δ 7.30 (s, 2H), 7.10 (s, 2H), 3.44 (s, 2H), 3.36 (dd, 2H, J₁=17.5 Hz, J₂=5.70 Hz), 2.87 (d, 2H, J=17.3 Hz), 2.16 (s, 2H).

Synthesis of 5,6,11,12-tetrahydro-5,11-methanodibenzo[a,e][8]annulene-2,3,8,9-tetracarbonitrile (iv). 5,6,11,12-Tetrahydro-5,11-methanodibenzo[a,e][8]annulene-2,3,8,9-tetrayl tetrakis(trifluoromethanesulfonate) (4.71 g, 5.80 mmol), Pa₂(dba)₃ (600 mg, 10%), DPPF (600 mg) and Zn(CN)₂ (650 mg) were added to 30 mL absolute DMF. The mixture was degassed, flushed with N₂ for three times and then heated to 110° C. The clear dark brown solution was kept at 110° C. for 10 min and then another 3 portions of Zn(CN)₂ (650 mg, 650 mg, 650 mg) were added in 45 min The solution was then stirred for 10 min and poured into water (200 mL), washed with methanol and the remaining solid was loaded to a flash column using dichloromethane/ethyl acetate=5/1; the product was obtained as an off-white solid (1.67 g, 90% yield). TLC: dichloromethane, R_{f}=0.2; ¹H NMR (500 MHz, CDCl₃): δ 7.68 (s, 2H), 7.46 (s, 2H), 3.56 (s, 2H), 3.45 (dd, 2H, J₁=22.2 Hz, J₂=7.00 Hz), 2.96 (d, 2H, J=21.9 Hz), 2.25 (s, 2H).

Synthesis of 5,6,11,12-tetrahydro-5,11-methanodibenzo[a,e][8]annulene-2,3,8,9-tetracarboxylic acid (v). The intermediate iv (320 mg, 1.00 mmol) was dispersed in ethanol (6 mL). To it, KOH (1.16 g, 20.0 mmol) dissolved in water (6 mL) was added to the mixture dropwise in 10 mins. The system was refluxed for 12 h and then the ethanol was removed by rota-evaporation. The solution was cooled to room temperature and acidified using HCl (6N) to adjust the pH between 1˜2. A large quantity of white precipitate was formed, filtrated and washed with dilute HCl (2N) and then with water for two times. An off-white solid (385 mg, yield: 97.2%) was obtained by drying the solid in a vacuum oven at 50° C. for 24 h. ¹H NMR (500 MHz, DMSO-d₆): 12.9 (s, 4H), 7.57 (s, 2H), 7.25 (s, 2H), 3.43 (s, 2H), 3.25 (m, 2H), 2.85 (d, 2H, J=21.4 Hz), 2.09 (s, 2H).

Synthesis of the carbocyclic pseudo Tröger's base-based dianhydride CTB1 (vi). 5,6,11,12-Tetrahydro-5,11-methanodibenzo[a,e][8]annulene-2,3,8,9-tetracarboxylic acid (v), 200 mg, 0.51 mmol) was added to acetic anhydride (15 mL), which was heated to reflux and kept for 1 h. The solution was then cooled to room temperature and a large quantity of needle crystals were filtrated, washed with cold acetic anhydride and dried in a vacuum oven at 140° C. for 24 h. Off-white needle crystals (174 mg, yield: 95%) were obtained and were used without further purification. ¹H NMR (700 MHz, CDCl₃): δ 7.88 (s, 2H), 7.62 (s, 2H), 3.69 (s, 2H), 3.56 (d, 2H, J=17.7 Hz), 3.11 (d, 2H, J=17.6 Hz), 2.31 (s, 2H). ¹³C NMR (175 HZ, CDCl₃): δ 162.66, 162.47, 149.28, 143.25, 129.63, 129.60, 126.87, 126.39, 40.28, 33.09, 27.22; mp: 375.7° C.; HRMS for [M+H⁺, C₂₁H₁₃O₆⁺, ESI]; Calcd. for 361.0707; Found: 361.0707; Elementary analysis: Calcd. for C₂₁H₁₂O₆: C, 70.00; H, 3.36; Found: C, 70.33; H, 3.55.

Synthesis of 6,12-dioxo-5,6,11,12-tetrahydro-5,11-methanodibenzo[a,e][8]annulene-2,3,8,9-tetraOH (viii). 6,12-Dioxo-5,6,11,12-tetrahydro-5,11-methanodibenzo[a,e][8]annulene-2,3,8,9-tetramethoxy ether (2.00 g, 5.43 mmol, vii) was dissolved in 150 mL dichloromethane and cooled in an ice bath. To it, BBr₃ (3.1 mL, 32.6 mmol) was added to the solution dropwise. After the solution was stirred at room temperature for 24 h and then poured into 200 g crushed ice, an off-white powder was obtained after stirring under N₂ for another 24 h. After filtration, the solid was washed 4 times with water and dried in a vacuum oven at 60° C. for 24 h. (1.61 g, yield: 95%). The product was used directly for further reactions.

Synthesis of 6,12-dioxo-5,6,11,12-tetrahydro-5,11-methanodibenzo[a,e][8]annulene-2,3,8,9-tetrayl tetrakis(trifluoromethanesulfonate) (ix). 6,12-Dioxo-5,6,11,12-tetrahydro-5,11-methanodibenzo[a,e][8]annulene-2,3,8,9-tetraOH (2.00 g, 6.41 mmol, viii) and triethylamine (13.76 g, 128.0 mmol) were added to dichloromethane (150 mL) and cooled in an ice bath. To it, triflic anhydride (32.0 g, 128.0 mmol) was added dropwise. The reaction system was further stirred for 12 h and poured into ice water (300 mL). The water phase was then extracted twice with dichloromethane (2×30 mL). The organic phase was combined and dried with magnesium sulfate. The solution was removed by rota-evaporation and loaded to a column packed with silica gel. An off-white product (3.23 g, yield: 60%) was obtained after column chromatography. ¹H NMR (500 MHz, CDCl₃): δ 8.10 (s, 2H), 7.63 (s, 2H), 4.18 (s, 2H), 3.09 (s, 2H). ¹³C NMR (125 MHz, CDCl₃): δ 189.1, 144.4, 141.2, 140.5, 129.3, 124.1, 119.4, 117.6, 47.1, 30.9.

Synthesis of 6,12-dioxo-5,6,11,12-tetrahydro-5,11-methanodibenzo[a,e][8]annulene-2,3,8,9-tetracarbonitrile (x). 5,11-Methanodibenzo[a,e][8]annulene-6,12(5H,11H)-dione-2,3,8,9-tetratriflic ester (4.71 g, 5.88 mmol, ix), Pa₂(dba)₃ (600 mg, 10%), DPPF (600 mg) and Zn(CN)₂ (650 mg) were added to 30 mL absolute DMF. The mixture was degassed, flushed with N₂ and then heated to 110° C. The clear dark brown solution was kept at 110° C. for 10 min and then another 3 portions of Zn(CN)₂ (650 mg, 650 mg, 650 mg) were added in 45 min. The solution was then stirred for 10 min and poured into water (200 mL), washed with methanol and the remaining solid was loaded to a flash column using dichloromethane/ethyl acetate=5/1; the product was obtained as an off-white solid (1.5 g, 76% yield). TLC: Dichloromethane; R_{f}=0.15; ¹H NMR (500 MHz, DMSO-d₆): 8.45 (s, 2H), 8.34 (s, 2H), 4.33 (s, 2H), 3.08 (s, 2H). ¹³C NMR (125 MHz, DMSO-d₆): δ 190.4, 144.3, 135.4, 133.4, 132.2, 119.5, 115.7, 115.4, 115.5, 47.5, 29.4.

Synthesis of 6,12-dioxo-5,6,11,12-tetrahydro-5,11-methanodibenzo[a,e][8]annulene-2,3,8,9-tetracarboxylic acid (xi). 6,12-Dioxo-5,6,11,12-tetrahydro-5,11-methanodibenzo[a,e][8]annulene-2,3,8,9-tetra-carbonitrile (110 mg, 0.316 mmol, x) was dissolved in anhydrous H₂SO₄, (4 mL), To it, water (4 mL) was added dropwise in 15 min. The system was further heated to reflux for 24 h. The resulting off-white crystalline precipitate was filtrated and washed with dilute HCl (2N) and then with water (20 mL) twice. The pure intermediate xi was obtained after drying in a vacuum oven at 40° C. for 8 h. ¹H NMR (500 MHZ, DMSO-d₆): δ 13.5 (s, 4H), 8.10 (s, 2H), 7.68 (s, 2H), 4.23 (s, 2H), 3.03 (s, 2H).

Synthesis of the carbocyclic pseudo Tröger's base-based dianhydride CTB2 (xii). The tetra-acid intermediate (200 mg, 0.471 mmol, xi) was added to acetic anhydride (15 mL), which was heated to reflux and kept for 1 h. The solution was then cooled to room temperature and a large quantity of needle crystals were filtrated, washed with cold acetic anhydride and dried in a vacuum oven at 140° C. for 24 h. Off-white needle crystals (174 mg, yield: 95%) were obtained and were used without further purification. ¹H NMR (700 MHz, CDCl₃): δ 8.63 (s, 2H), 8.19 (s, 2H), 4.45 (s, 2H), 3.14 (s, 2H). ¹³C NMR (175 MHz, CDCl₃): δ 189.6, 161.0, 160.9, 146.4, 135.4, 134.6, 131.6, 126.8, 126.6, 48.9, 30.1; mp: 318.5° C.; HRMS for [M+H⁺, C₂₁H₉O₈⁺, ESI]; Calcd. for: 389.0292; Found: 389.0292; Anal: Calcd. for C₂₁H₈O₈: C, 64.96; H, 2.08; Found: C, 64.57; H, 2.27.

Synthesis of CTB1-DMN. CTB1 (95.9 mg, 0.2665 mmol, vi) and 3,3′-dimethylnaphthidine (DMN, 84.9 mg, 0.2665 mmol) were added to m-cresol (1.2 mL) in a Schlenk tube. The system was stirred at room temperature under N₂ atmosphere for 15 min and then heated to 60° C. for half an hour and a clear solution was formed. One drop of isoquinoline was added to the solution which was heated to 180° C. for 4 h to form a viscous solution. The solution was then cooled to room temperature and precipitated in methanol. The solid was re-dissolved in chloroform and re-precipitated in methanol twice. The polymer was obtained as an off-white filament with a yield of 95%. Tₐ=520° C., ¹H NMR (500 MHz, CDCl₃): δ 8.01 (s, 2H), 7.76 (s, 2H), 7.53-7.63 (m, 8H), 7.32 (s, 2H), 3.76 (s, 2H), 3.64 (s, 2H), 3.224 (s, 2H), 2.39-2.43 (m, 8H). FT-IR (wavenumber, cm⁻¹): 2928 (m, asy of C—H), 1776, 1699, 1616 cm⁻¹ (s, C═O stretching), 1373 (s, Ar stretching), 869, 741 (s, C—N vibration), Mₙ=5.90×10⁴ g mol⁻¹; PDI=1.52; S_{BET}=580 m² g⁻¹; Anal. Calcd. for C, 80.86; H, 4.73; N, 4.39; Found: C, 78.74; H, 4.51; N, 4.12.

Synthesis of CTB2-DMN. CTB2 (103.4 mg, 0.2665 mmol, xii) and 3,3′-dimethylnaphthidine (DMN, 84.9 mg, 0.2665 mmol) were added to m-cresol (1.2 mL) in a Schlenk tube. The system was stirred at room temperature under N₂ atmosphere for 15 min and then heated to 60° C. for half an hour and a clear solution was formed. One drop of isoquinoline was added to the solution which was heated to 180° C. for 4 h to form a viscous solution. The solution was then cooled to room temperature and precipitated in methanol. The solid was re-dissolved in DMF and re-precipitated in methanol twice. A light yellow solid (170 mg, yield: 95.5%) was obtained after drying in a vacuum oven at 120° C. for 24 h. ¹H NMR (700 MHz, DMSO-d₆): δ 8.41 (s, 2H), 8.30 (s, 2H), 7.91 (s, 2H), 7.71 (s, 2H), 7.30 -7.50 (m, 6H), 4.69 (s, 2H), 3.30 (s, 2H), 2.33 (m, 6H); FT-IR (wavenumber, cm⁻¹): 2923 (m, asy of C—H), 1782, 1717, 1616 cm⁻¹ (s, C═O stretching), 1394 (s, Ar stretching), 872, 811(s, C—N vibration); Tₐ=480° C. S_{BET}=489 m² g⁻¹; Mₙ=2.0×10⁴ g mol⁻¹; PDI=1.63; Anal. Calcd. for C, 77.47; H, 3.93; N, 4.20; Found: C, 72.76; H, 3.63; N, 3.55.

Film Preparation. Polymer solutions (3 wt/vol %) of CTB1-DMN in CHCl₃ and CTB2-DMN in DMF were filtered through 0.45 μm PTFE filters and poured onto flat glass Petri dishes. The CTB1-DMN solution was slowly evaporated at room temperature for one day. The CTB2-DMN solution was evaporated at 70° C. in an oven for one day. Thereafter, the obtained polymer films were further dried at 120° C. for 6 h under vacuum. To remove any traces of residual solvent, both membrane types were soaked in methanol for 24 h, air-dried, and then post-dried at 120° C. in a vacuum oven for 24 h. Complete solvent removal from the polymer films was confirmed by TGA.

Gas permeation measurements. The gas permeability of the polymers was determined using the constant-volume/variable-pressure method. The isotropic films were degassed in the permeation system on both sides under high vacuum for at least 24 h. The increase in permeate pressure with time was recorded by a MKS Baratron transducer. The permeability of all gases was measured at 2 bar upstream pressure at 35° C. by:

$$P = D \times S = 1 {0}^{10} \times \frac{{V}_{d} \times l}{{p}_{u ρ} \times T \times R \times A} \times \frac{d p}{d t} \qquad ( 2 )$$

where P is the permeability (Barrer)−1 Barrer=10⁻¹⁰ cm³(STP)·cm/cm²·s·cmHg, pᵤₚ is the upstream pressure (cmHg), dp/dt is the steady-state permeate-side pressure increase (cmHg/s), V_{d} is the calibrated permeate volume (cm³), l is the membrane thickness (cm), A is the effective membrane area (cm²), T is the operating temperature (K), and R is the gas constant (0.278 cm³·cmHg/cm³(STP)·K). The apparent diffusion coefficient D (cm²/s) of the polymer membrane was calculated by D=l²/6θ, where l is the membrane thickness and θ is the time lag of the permeability measurement. The solubility coefficient S (cm³ (STP)/cm³·cmHg) was obtained from the relationship S=P/D.

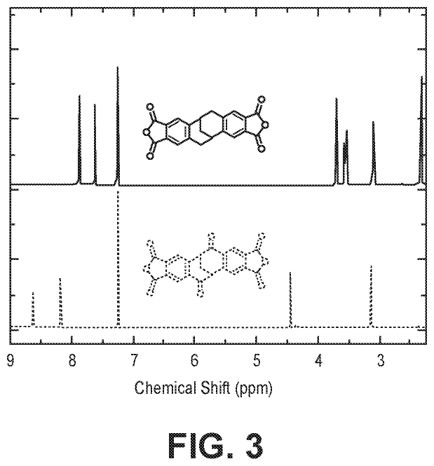

Synthesis and Physical Characterization. The carbocyclic pseudo Tröger's base-derived dianhydrides were synthesized by the following steps: first, the tetramethoxyethers (intermediate i and vii) were reacted with BBr₃ to obtain the corresponding biscatecol intermediates (ii and viii), which were then converted to the trifluoromethylsulfonic ester intermediates (iii and ix) by reaction with trifluoromethane sulfonic anhydride. The trifluoromethylsulfonic groups were substituted by cyano groups using Zn(CN)₂ under catalytic amount of Pd₂(dba)₃ and DPPF as ligand to form the tetracyano intermediate (iv and x). Similar reaction schemes were previously reported by our group for triptycene-based trifluoromethylsulfonic ester intermediates. The hydrolysis of intermediate (iv) was conducted using a KOH/water/ethanol system and the resulting tetra-acid (v) was obtained in quantitative yield. Unlike the previously reported hydrolysis of tetracyano-substituted intermediates to the corresponding acids under basic conditions, in this work the intermediate (x) was hydrolyzed to its corresponding tetra-acid intermediate xi using 50% sulfuric acid. Under basic conditions, the dione group in the carbocyclic kink affected the hydrolysis reaction and based on NMR results yielded multiple products. The dianhydrides CTB1 (vi) and CTB2 (xii) were obtained by refluxing the tetra-acid intermediates with acetic anhydride. After re-crystallization with acetic anhydride, needle dianhydride crystals were obtained. The structure of the dianhydrides was confirmed by their NMR spectra, FT-IR, HRMS and elemental analysis. Their proton NMR spectra are shown in FIG. 3. The strong electron withdrawing properties of the dione group had significant effect on the electronic properties of the dianhydride, as indicated by a significant low-field shift of the aromatic protons from 7.62˜7.88 ppm of CTB2 to 8.19˜8.63 ppm of CTB1.

The polyimides (Scheme 3) were obtained by reaction of the two dianhydrides CTB1 and CTB2, respectively, with 3,3-dimethylnaphthadine under catalytic amount of isoquinoline in m-cresol at 180° C. for 3 h under a continuous flow of N₂. CTB1-DMN demonstrated good solubility in NMP, m-cresol and chloroform, whereas CTB2-DMN was only soluble in DMF, NMP and m-cresol. The molecular weights of the polymers were obtained by GPC using narrow polydispersity polystyrene as external standard (Table 1). CTB1-DMN and CTB2-DMN had number average molecular weights of 59,000 g mol⁻¹ and 20,000, respectively, with narrow polydispersity index (PDI) of ˜1.5-1.6.

TABLE 1
Basic Properties of the CTB-Based PIM-PIs

| Polymer | Mₙ (g mol⁻¹)^{a} | PDI | S_{BET} (m² g⁻¹) | T_{d} (° C.) | ρ (g cm⁻³) |
|---|---|---|---|---|---|
| CTB1-DMN | 5.9 × 10⁴ | 1.52 | 580 | 520 | 1.18 |
| CTB2-DMN | 2.0 × 10⁴ | 1.62 | 469 | 480 | 1.20 |

^{a}The molecular weights were obtained using chloroform (CTB1-DMN) and DMF (CTB2-DMN) as solvents, respectively.

TABLE 2
BET surface area and pore volume of CTB1-DMN and CTB2-DMN

| | S_{BET} | S_{BET} | Pore | Pore |
|---|---|---|---|---|
| | m²/g^{a} | m²/g^{b} | volume^{c} | volume^{d} |
| Polymer | (N₂) | (CO₂) | (N₂) | (CO₂) |
| CTB1-DMN | 580 | 502 | 0.363 | 0.120 |
| CTB2-DMN | 469 | 613 | 0.291 | 0.145 |

^{a}Surface area was obtained by N₂ adsorption from relative pressure of 0.05 to 0.30.
^{b}Surface area is obtained by CO₂ adsorption; the cumulative surface area was 3.3 Å-7.2 Å;
^{c}Pore volume obtained by N₂ adsorption;
^{d}Pore volume was obtained by CO₂ adsorption up tol bar.

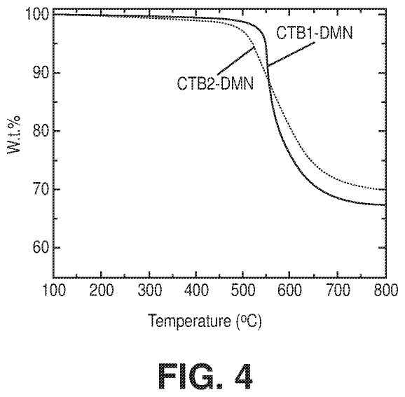

Both PIM-PIs demonstrated excellent thermal stability (FIG. 4, Table 1) with onset decomposition temperatures of 520 and 480° C. for CTB1-DMN and CTB2-DMN, respectively.

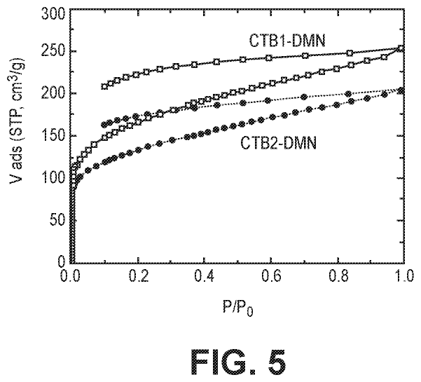

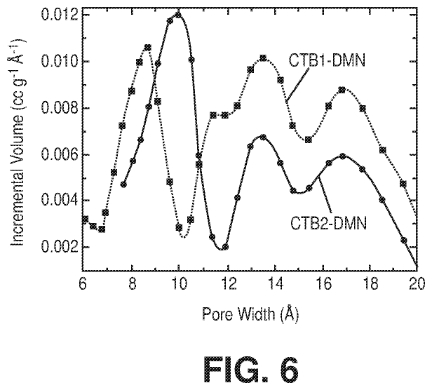

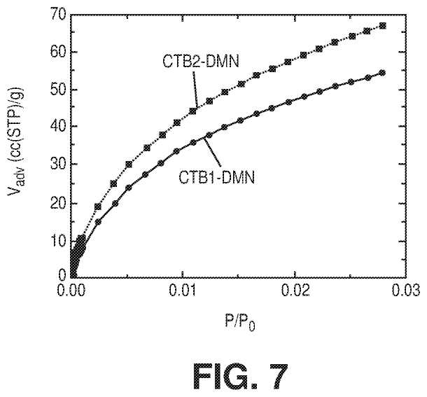

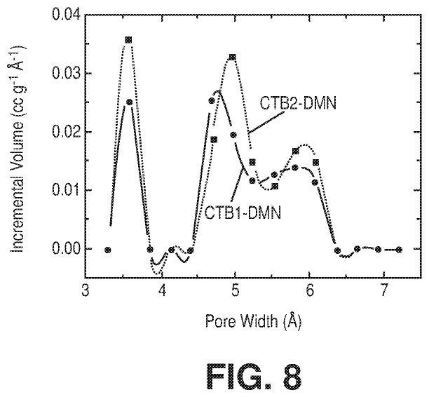

The two polyimides showed high microporosity as demonstrated by their N₂ (−196° C.) adsorption isotherms, as shown in FIG. 5. The BET surface areas of CTB1-DMN and CTB2-DMN were 580 m² g⁻¹ and 469 m² g⁻¹, respectively. FIGS. 5-8 are graphical views of N₂ and CO₂ isotherms of polymers based on pseudo TB-derived dianhydrides and pore size distributions. In particular, FIG. 6 is a graphical view of pore size distribution based on nitrogen adsorption showing incremental volume (cc g⁻¹ Å⁻¹) versus pore width (Å) for CTB1-DMN and CTB2-DMN, according to one or more embodiments of the present disclosure. FIG. 7 is a graphical view of CO₂ volume adsorbed (cc(STP)/g) versus P/P₀ for CTB1-DMN and CTB2-DMN, according to one or more embodiments of the present disclosure. FIG. 8 is a graphical view of pore size distribution based on CO₂ adsorption showing incremental volume (cc g⁻¹ Å⁻¹) versus pore width (Å) for CTB1-DMN and CTB2-DMN, according to one or more embodiments of the present disclosure.

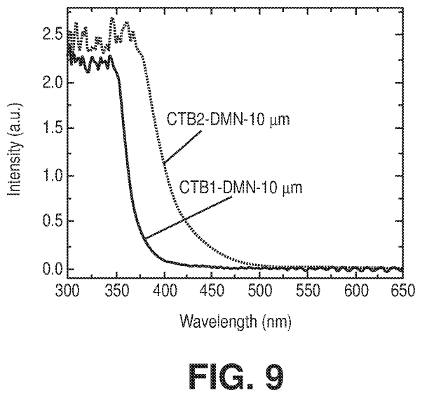

Previous studies demonstrated that the interchain packing of polyimides can be strongly influenced by charge-transfer complex (CTC) formation, which has significant effects on their gas transport properties. The strength of CTC interactions follow a qualitative trend with a red shift in the wavelength. To elucidate the differences in CTC formation between the two polyimides, their UV-vis spectra were measured using 10-μm-thick films (FIG. 9). It is clear that the dione-containing CTB2-DMN film exhibited stronger CTC interactions as compared to the CTB1-DMN analogue, as the cut-off wavelength was 550 nm for CTB2-DMN, whereas that of CTB1-DMN was around 420 nm.

Gas permeation properties of the CTB1-DMN and CTB2-DMN PIM-PIs. The gas permeation properties of mechanically strong CTB -based polyimide films were determined by the constant-volume/variable-pressure technique. The results are summarized in Table 3. The gas permeation properties of previously reported related PIM-PIs derived from DMN with sterically hindered spirobisindane—(SBI), spirobifluorene—(SBF) and triptycene—(TRIP) dianhydrides are included in Table 3 for comparison. The fresh CTB-based PIM-PIs films showed high gas permeabilities with moderately high gas-pair selectivities. The dione-based CTB2-DMN polyimide exhibited lower gas permeability and higher selectivity values compared to the CTB1-DMN polyimide. For example, the O₂ permeabilities of CTB1-DMN and CTB2-DMN were 320 and 206 Barrer with O₂/N₂ selectivities of 4.2 and 5.2, respectively. This trend resulted from tighter chain packing in the CTB2-DMN polyimide due to stronger CTC formation and lower BET surface area, as discussed above. Upon physical aging over 60 days, gas permeabilities decreased significantly by 40-50% for both polyimides, which is a typical trend for intrinsically microporous polymers due to densification of the poorly packed glassy polymer chains. On the other hand, aging resulted in moderate increase in the gas-pair selectivities. Interestingly, the aged dione-based CTB2-DMN polyimide showed commendable performance for CO₂/CH₄ separation with CO₂ permeability of 546 Barrer and CO₂/CH₄ selectivity of 28.9. For comparison, the most prominent commercial membrane material for CO₂ removal from natural gas, cellulose triacetate, shows about the same CO₂/CH₄ selectivity (32.8) but with ˜80-fold lower CO₂ permeability (6.6 Barrer).

It is noteworthy to compare the gas permeation properties of the CTB-DMN-based PIM-PIs with those of other DMN-derived polyimides made from alternative dianhydrides containing sterically hindered PIM motif building blocks (Table 3). In general, the gas permeabilities of fresh DMN-based PIM-PIs follow the order P_{CH4}<P_{N2}<P_{O2}<P_{H2}<P_{CO2}, which is typical for highly permeable and low-to-moderately selective PIM-PIs. Interestingly, the permeability sequence for the dione-based CTB2-DMN indicates a more size-selective microporous structure as P_{CH4}˜P_{N2}<P_{O2}<P_{CO2}<PH₂. This trend has been ascribed to the presence of size-selective ultramicropores (<7 Å) in previously reported PIM-PIs that have defined the 2015 permeability/selectivity upper bounds for O₂/N₂, H₂/CH₄ and H₂/N₂ separations.

TABLE 3
Gas Permeability and Selectivity of CTB1-DMN and CTB2-DMN Polyimides.
Related DMN-Derived PIM-PIs with Various PIM-Motif Dianhydrides are Listed for Comparison.

| | Permeability (Barrer) | | | | | Ideal selectivity (α_{X/Y}) | | | |
|---|---|---|---|---|---|---|---|---|---|
| Polymer | H₂ | N₂ | O₂ | CH₄ | CO₂ | H₂/N₂ | H₂/CH₄ | O₂/N₂ | CO₂/CH₄ |
| CTB1-DMN^{a} | 1,295 | 76.2 | 320 | 95.7 | 1,661 | 17.0 | 13.5 | 4.2 | 17.4 |
| Aged 60 d | 759 | 32.4 | 152 | 36.2 | 795 | 23.4 | 21.0 | 4.7 | 24.5 |
| CTB2-DMN^{b} | 1,150 | 39.9 | 206 | 40.4 | 948 | 28.8 | 28.5 | 5.2 | 23.5 |
| Aged 60 d | 737 | 19.7 | 106 | 18.9 | 546 | 37.5 | 39.1 | 5.4 | 28.9 |
| TDA1-DMN^{c} | 3,047 | 182 | 783 | 216 | 3,700 | 17.0 | 14.1 | 4.3 | 17.0 |
| Aged 250 d | 2,430 | 134 | 609 | 158 | 3,000 | 18.0 | 15.4 | 4.5 | 19.0 |
| TDAi3-DMN^{c} | 2,233 | 160 | 594 | 211 | 3,154 | 14.0 | 10.6 | 3.7 | 14.9 |
| Aged 250 d | 2,114 | 130 | 505 | 170 | 2,670 | 16.2 | 12.4 | 3.9 | 15.7 |
| SBIDA-DMN^{d} | 840 | 94 | 295 | 170 | 2,180 | 8.9 | 4.9 | 3.1 | 12.8 |
| (PIM-PI-10) | | | | | | | | | |
| SBFDA-DMN^{e} | 2,966 | 226 | 850 | 326 | 4,700 | 13.1 | 9.1 | 3.8 | 14.4 |
| Aged 200 days | 878 | 33 | 161 | 40 | 703 | 26.6 | 22.0 | 4.9 | 17.6 |
| EADA-DMN^{f} | 4,230 | 369 | 1,380 | 457 | 7,340 | 11.5 | 9.3 | 3.7 | 16.1 |
| (PIM-PI-12) | | | | | | | | | |
| Aged 273 days | 2,860 | 131 | 659 | 156 | 3,230 | 21.8 | 18.3 | 5.0 | 20.7 |

^{a}Freshly made CTB1-DMN soaked in MeOH for 24 h and then dried at 120° C. in a vacuum oven for 24 h; membrane thickness was 48 μm.
^{b}Freshly made CTB2-DMN soaked in MeOH for 24 h and then dried at 120° C. in a vacuum oven for 24 h, membrane thickness was 57 μm.
^{c}Data from reference 36; the membranes were soaked in MeOH for 24 h and then dried at 120° C. in a vacuum oven for 24 h; membrane thickness was ~85-100 μm.
^{d}Data from reference 18; the permeability of the as-cast membrane tested by GC method.
^{e}Data from reference 20, SBFDA-DMN membrane was soaked in MeOH for 24 h and then dried at 120° C. in a vacuum oven for 24 h; membrane thickness was 129 μm;
^{f}Data from reference 19; the film was soaked in methanol for 8 h and then dried in air; membrane thickness was 72 μm.

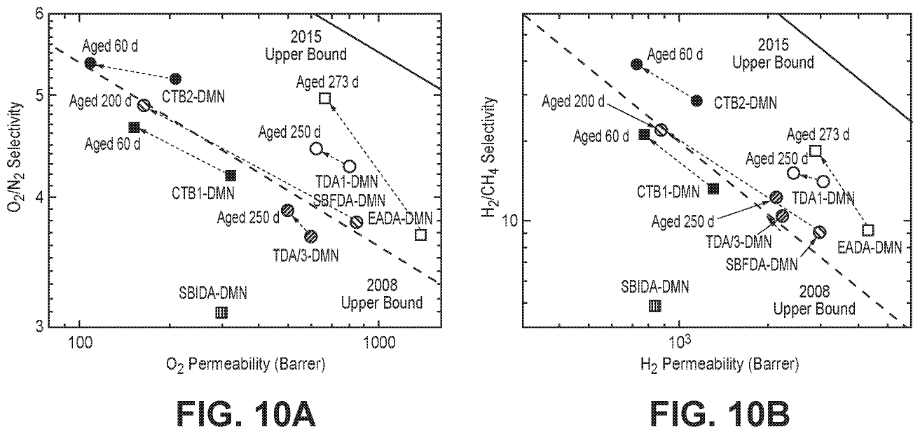

The performance of the CTB-DMN-based PIM-PIs for O₂/N₂ and H₂/CH₄ separation relative to the 2008 upper bounds is shown in FIGS. 10a-10b and compared to related DMN-based PIM-PIs. In this PIM-PI series, CTB2-DMN displayed the highest selectivity with lower permeability than PIM-PIs derived from dianhydrides bearing alternative sites of contortion, such as TDA, EADA, SBFDA and SBIDA. Compared to conventional low-free-volume glassy polymers used for commercial O₂/N₂ separation, such as polysulfone, aged CTB2-DMN showed similar O₂/N₂ selectivity with about 80-fold higher permeability.

Accordingly, this Example demonstrated that carbocylic pseudo Tröger's base dianhydrides, specifically CTB2, are promising new building blocks for the synthesis of high-performance PIM-PIs for membrane gas separation applications. Further optimization of CTB-based PIM-PIs with functionalized diamines to enhance selectivity could further broaden the commercial prospects of this novel polyimide platform.

In sum, two novel carbocyclic pseudo Trögef s base-derived polyimides of intrinsic microporosity were synthesized by polycondensation reaction of 5,6,11,12-tetrahydro-5,11-methanodibenzo[a,e][8]annulene-2,3,8,9-tetracarboxylic anhydride (CTB1) or its dione-substituted analogue 6,12-dioxo-5,6,11,12-tetrahydro-5,11-methanodibenzo[a,e][8]annulene-2,3,8,9-tetracarboxylic dianhydride (CTB2) with 3,3′-dimethylnaphthidine (DMN). Both CTB1-DMN and CTB2-DMN showed excellent thermal stability and significant microporosity as demonstrated by high BET surface areas of 580 and 469 m² g⁻¹, respectively. Compared to related DMN-based PIM-PIs made from dianhydrides with different contortion sites (SBI, SBF, TDA, EADA), CTB2-DMN showed higher gas-pair selectivities and lower permeabilities. The excellent balance between high permeability and high pair selectivity makes CTB2-DMN a promising membrane material with performance located between the 2008 and 2015 permeability/selectivity upper bounds for O₂/N₂ and H₂/CH₄.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A pseudo Tröger's Base (TB)-based dianhydride, comprising:
a pseudo TB-based dianhydride characterized by one of the following chemical structures: 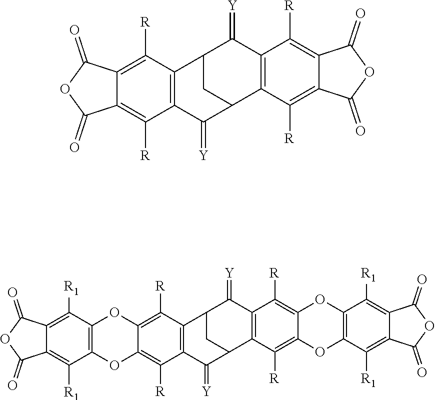
where Y is O, CH₂, or H₂ and each R and R₁ is independently any aromatic group or any aliphatic group.

2. The dianhydride of claim 1, wherein the pseudo TB dianhydride is a carbocyclic pseudo TB dianhydride.

3. The dianhydride of claim 1, wherein the pseudo TB dianhydride is a building block for the synthesis of microporous polymers.

4. The dianhydride of claim 3, wherein the microporous polymers include a microporous polyimide.

5. The dianhydride of claim 1, wherein each R and R₁ is independently selected from methyl, ethyl, propyl, isopropyl, n-butyl, and iso-butyl.

6. The dianhydride of claim 1, wherein the pseudo TB-based dianhydride is one or more of the following:

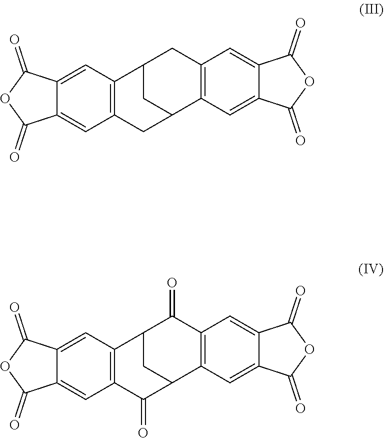 [SMILES: O=C1OC(=O)c2cc3c(cc21)CC1CC3Cc2cc3c(cc21)C(=O)OC3=O ; O=C1OC(=O)c2cc3c(cc21)C(=O)C1CC3C(=O)c2cc3c(cc21)C(=O)OC3=O]

7. A polyimide, comprising:
a polyimide characterized by one or more of the following chemical structures: 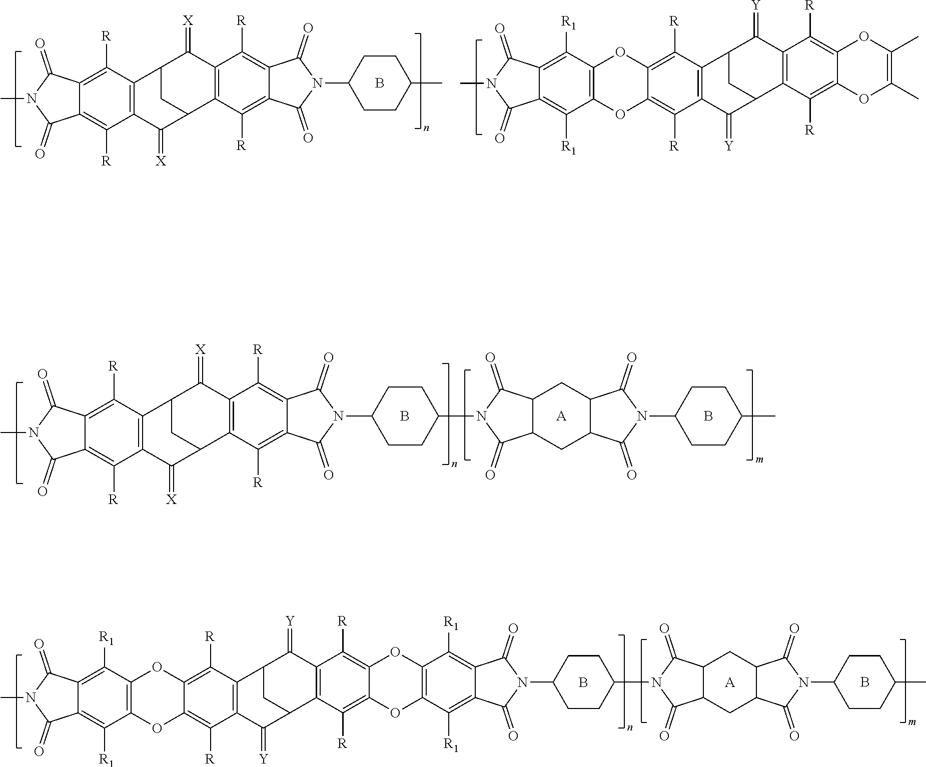
where Y is O, CH₂, or H₂; each R and R₁ is independently any aromatic group or aliphatic group; A is any dianhydride; B is any diamine; and n and/or m ranges from 1 to 10,000.

8. The polyimide of claim 7, wherein the polyimide is a microporous polyimide.

9. The polyimide of claim 7, wherein the polyimide is derived from a carbocyclic pseudo TB-derived dianhydride.

10. The polyimide of claim 7, wherein the polyimide is soluble in organic solvents.

11. The polyimide of claim 10, wherein the organic solvents include one or more of NMP, m-cresol, DMF, and chloroform.

12. The polyimide of claim 7, wherein a molecular weight of the PIM-PI or polyimide ranges from about 20,000 g mol⁻¹ to about 60,000 g mol⁻¹.

13. The polyimide of claim 7, wherein a polydispersity index ranges from about 1.5 to about 1.6.

14. The polyimide of claim 7, wherein each R and R₁ is independently selected from methyl, ethyl, propyl, isopropyl, n-butyl, and iso-butyl.

15. The polyimide of claim 7, wherein A is selected from one of the following:

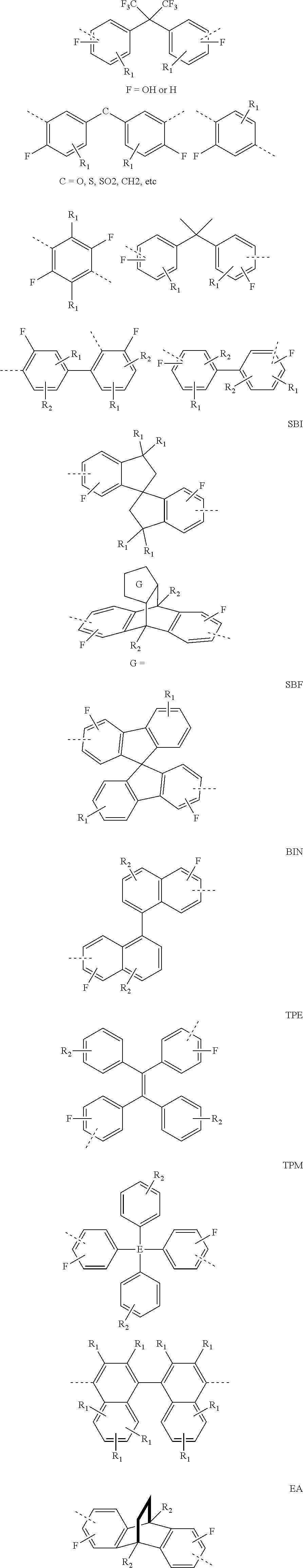

16. The polyimide of claim 7, wherein B is selected from one of the following:

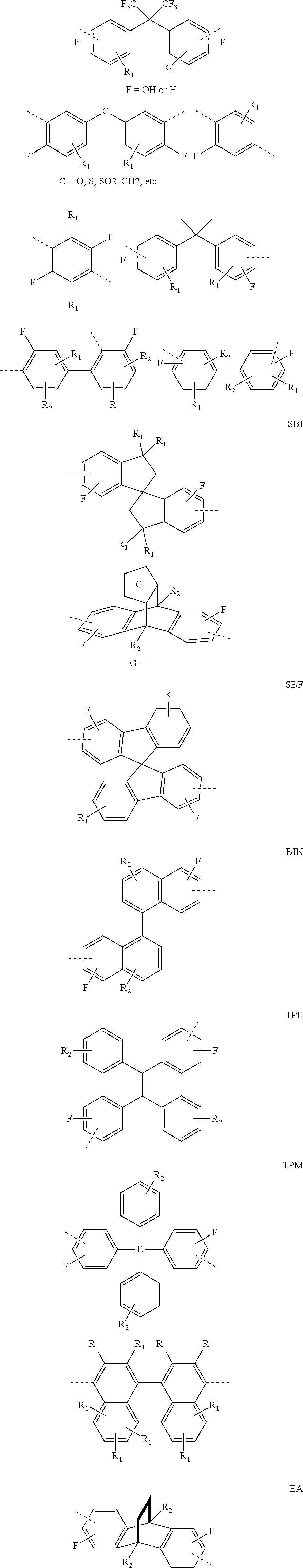

17. The polyimide of claim 7, wherein the PIM-PI or polyimide is fabricated into a polymeric membrane.

18. A method of separating chemical species in a fluid composition, comprising:
contacting a polyimide-based membrane with a fluid composition including at least two chemical species, wherein the polymeric membrane includes a polyimide characterized by one or more of the following chemical structures: 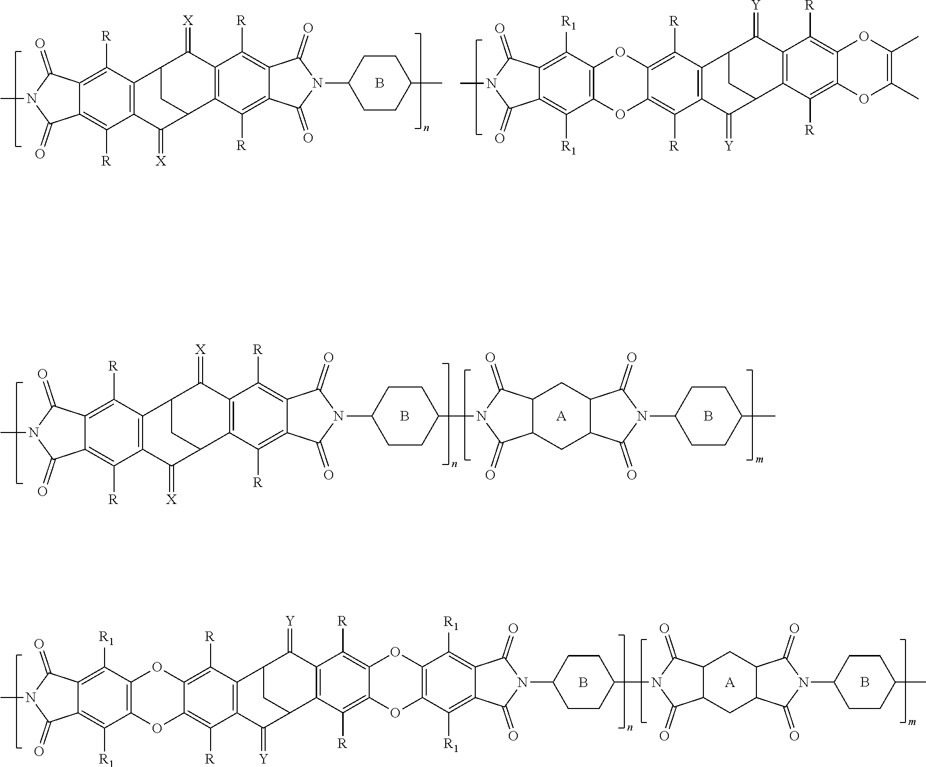
and capturing at least one of the chemical species from the fluid composition.

19. The method of claim 18, wherein the chemical species of the fluid composition includes two or more of O₂, N₂, H₂, He, CO₂, aliphatic C₁₊ hydrocarbons, olefins, paraffins, xylene isomers, n-butane, iso-butane, and butenes.

20. The method of claim 18, wherein the captured chemical species includes one or more of H₂, O₂, and CO₂.
